(19) United States
(12) Patent Application Publication
(10) Pub. No.: US 2025/0290093 A1
(43) Pub. Date: Sep. 18, 2025

(54) HIGH CAPACITY LENTIVIRAL VECTORS

(71) Applicants: CZ Biohub SF, LLC, San Francisco, CA (US); The Board of Trustees of the Leland Stanford Junior University, Stanford, CA (US)

(72) Inventors: Michael A. Fischbach, Stanford, CA (US); Gabriel T. Filsinger, Palo Alto, CA (US); Brian J. Caliando, Redwood City, CA (US)

(73) Assignees: CZ Biohub SF, LLC, San Francisco, CA (US); The Board of Trustees of the Leland Stanford Junior University, Stanford, CA (US)

(21) Appl. No.: 18/861,440

(22) PCT Filed: May 1, 2023
(86) PCT No.: PCT/US2023/020566

§ 371 Date: Oct. 29, 2024

Related U.S. Application Data
(60) Provisional application No. 63/336,691, filed on Apr. 29, 2022.

(51) Int. Cl. C12N 15/86

(52) U.S. Cl. CPC C12N 15/86; C12N 2740/15022; C12N 2740/15043; C12N 2740/15052; C12N 2800/40

(57) ABSTRACT

Described herein are lentiviral vectors and lentiviruses (comprising one or more lentivirus particles) with a payload capacity that is roughly double that of existing lentiviral vectors and lentiviruses. Kits and methods for making and using such vectors and viruses are also provided according to the present disclosure.

### CROSS-REFERENCE TO RELATED APPLICATIONS

This application claims priority to. and the benefit of. co-pending U.S. Provisional Application No. 63/336,691 entitled “HIGH CAPACITY LENTIVIRAL VECTORS” and filed on Apr. 29, 2022, the contents of which are incorporated in its entirety by reference as if fully set forth herein.

### REFERENCE TO A SEQUENCE LISTING SUBMITTED AS XML VIA PATENT CENTER

The instant application contains a Sequence Listing that has been filed electronically in.xml format and is hereby incorporated by reference in its entirety. Said.xml copy, created on May 1, 2023, is named “007610WO_1374026_Sequence_Listing.xml” and is 16 kilobytes in size.

### BACKGROUND

For cell and gene therapy, genetic material may be delivered to cells by a viral vector. However, the small payload capacity of currently available vectors limits the problems that can be solved using this approach. For example, Lentiviral vectors (LVVs) are limited by a payload capacity of about 8-12 kb. Thus, the LVV particle typically accommodates only 1-2 genes, making LVVs unsuitable for a broad range of valuable biotechnological applications in both the research and clinical spheres.

Lentiviral vectors may be derived from a number of Lentiviruses. The most commonly used vectors are derived from HIV-1, but LVVs may be derived from other lentiviruses, such as HIV-2, SIV_{SM}, SIV_{AGM}, EIAV, FIV, VNV, VAEV and BIV. See Durand S and Cimarelli A., 2011, “The inside out of lentiviral vectors,” Viruses 3(2):132-159. doi:10.3390/v3020132; see Milone, M. and O'Doherty, U. 2018, “Clinical use of lentiviral vectors” Leukemia 32.7:1529-1541, both incorporated herein by reference.

Production of LVV particles generally involves introducing three or four DNA plasmids into a mammalian cell culture (e.g., 293T cells). Producer cells transfected with each of the three, or each of the four, plasmids produce viral particles that can be recovered from the culture supernatant. In three-plasmid systems one of the plasmids (“transfer plasmid”) encodes an RNA transcript that includes an expression cassette comprising the vector “cargo” (e.g., sequences encoding the protein(s) or nucleic acid(s) of interest linked to a promoter(s)). The transfer plasmid also includes sequences required for dimerization (e.g., DIS and PSI) and along with flanking long terminal repeat (LTR) sequences. Illustrative transfer plasmids are shown in FIG. 1. A second plasmid (“envelope plasmid”) comprises a sequence encoding the envelope protein Env and a promoter operably linked to the coding sequence. A third plasmid (“packaging plasmid”) comprises a sequence encoding the Gag, Pol, Tat and Rev proteins, and a promoter operably linked to the coding sequence. In four-plasmid systems, two packaging plasmids (one encoding Rev and one encoding Gag and Pol) are used. In four-plasmid systems, Tat is replaced by a chimeric 5′ LTR fused to a heterologous promoter on the transfer plasmid. See “Lentiviral Guide,” (addgene.org/guides/lentivirus/).

### SUMMARY

Described herein are compositions, methods, and kits related to high-capacity lentiviral vectors and lentiviruses.

In one aspect, described herein are lentiviral vectors. In certain aspects, lentiviral vectors as described herein can comprise a first gRNA encoding a first cargo and a second gRNA encoding a second cargo, wherein the first cargo and second cargo are not the same, wherein the first gRNA and second gRNA are not covalently linked, and wherein the first gRNA and second gRNA comprise sequences that reduce or de-stabilize homopairing. In some embodiments, the first gRNA and second gRNA can comprise sequences that increase or promote heteropairing. In some embodiments, the first gRNA can comprise an extended duplex sequence (EDS), wherein the EDS can comprise sequences corresponding to the dimer initiation signal (DIS) DIS, stem loop packaging sequence, splice donor (“SD”) and the AUG domains of HIV-1. In some embodiments, the second gRNA can comprise an extended duplex sequence (EDS), wherein the EDS can comprise one or more sequences corresponding to the DIS, stem loop packaging sequence, splice donor (“SD”) and AUG domains of HIV-1. In some embodiments, the first gRNA and the second gRNA can comprise stem-loop structures, and the stem structure adjacent to a dimer initiation signal (DIS) of the first gRNA and the second gRNA can be incompatible. In some embodiments, the stem-loop structures adjacent to the DIS can comprise sequences that reduce the thermodynamic favorability of homopairing and increase the favorability of heteropairing relative to corresponding sequences in native HIV-1. In some embodiments, the dimer initiation signal (DIS) of the first gRNA and/or the second gRNA can be nonpalindromic sequences. In some embodiments, the first gRNA and/or the second gRNA, the SD domain may not bind with an H1 domain and/or the gag AUG initiation codon (AUG) domain may not bind with U5. In some embodiments, 5′LTR of the first gRNA and/or the 5′LTR of the second gRNA can be modified relative to the HIV-1 genome [SEQ ID NO:1] to increase the rate of heteropairing. In some embodiments, there can be a trans-activation responsive hairpin (TAR) hairpin, and the TAR of the first gRNA and/or second gRNA can be mutated or truncated relative to the TAR of the HIV-1 genome [SEQ ID NO:1]. In some embodiments, the 5′ LTR of either or both gRNAs can begin at the position corresponding to Position 2 of the HIV-1 gRNA sequence such that the cytosine base at Position 57 cannot be incorporated into the TAR hairpin stem.

In some embodiments, described herein are lentiviral vectors comprising a first gRNA (gRVA^{Long}) encoding a first cargo, the first cargo encoding at least one non-viral polymer being a non-viral polypeptide or a non-viral polynucleotide, optionally a bacterial polypeptide or polynucleotide, a eukaryotic polypeptide or polynucleotide, an animal polypeptide or polynucleotide, a plant polypeptide or polynucleotide, a mammalian polypeptide or polynucleotide, a human polypeptide or polynucleotide, an antibody, an inhibitory RNA; or a guide RNA, or combination thereof, and a second gRNA (gRNA^{Short}), wherein the first gRNA and second gRNA are not covalently linked, and wherein the second gRNA: does not encode a polymer selected from a bacterial polypeptide and a bacterial polynucleotide; and/or does not encode a polymer selected from a eukaryotic polypeptide and a eukaryotic polynucleotide; and/or does not encode a polymer selected from an animal polypeptide and an animal polynucleotide; and/or does not encode a polymer selected from a plant polypeptide and a plant polynucleotide; and/or does not encode a polymer selected from a mammalian polypeptide and a mammalian polynucleotide; and/or does not encode a polymer selected from a human polypeptide and a human polynucleotide; and/or does not encode an antibody; and/or does not encode an inhibitory RNA; and/or does not encode a guide RNA. In some embodiments, the second gRNA may not encode a polypeptide that is not a viral polypeptide. In some embodiments, the second gRNA may not encode a protein other than one or a combination of the following: VSVG, Gag, Rev, Tat, Vpr, Vpx, Vif and Nef. In some embodiments, the second gRNA may not encode a protein. In some embodiments, the first gRNA can comprise a payload at least 3 times larger, measured in kilobase pairs, than the payload of the second gRNA. In some embodiments, the payload of the first gRNA can be at least 4 times larger than the payload of the second gRNA. In some embodiments, the first gRNA can comprise a payload 3 times larger, measured in kilobase pairs, than the payload of the second gRNA. In some embodiments, the payload of the first gRNA can be 4 times larger than the payload of the second gRNA. In some embodiments, the payload flanked by the 5′ and 3′ LTRs of the first gRNA can be at least 5 times larger than the payload of the second gRNA. In some embodiments, the payload flanked by the 5′ and 3′ LTRs of the first gRNA can be 5 times larger than the payload of the second gRNA. In some embodiments, the first gRNA can comprise a payload at least 14 kb or more in size. In some embodiments, the combined payload of the first and second gRNAs can be less than 18 kb. In some embodiments, the first gRNA and second gRNA can comprise sequences that reduce or de-stabilize homopairing and increase or promote heteropairing.

In some embodiments, described herein are lentiviral vectors comprising a first gRNA (gRVA^{Long}) encoding a first cargo, the first cargo encoding at least one non-lentiviral polymer selected from a non-lentiviral polypeptide and a non-lentiviral polynucleotide, and a second gRNA (gRVA^{Short}) encoding a second cargo, wherein the first cargo and second cargo are not the same, wherein the first gRNA and second gRNA are not covalently linked, and wherein the payload flanked by the 5′ and 3′ LTRs of the first gRNA can be at least 3 times larger, measured in kilobase pairs, than the payload of the second gRNA. In some embodiments, described herein are lentivirus vector particles comprising a first gRNA (gRNA^{Long}) encoding a first cargo, the first cargo encoding at least one non-lentiviral polymer selected from a non-lentiviral polypeptide and a non-lentiviral polynucleotide, and a second gRNA (gRNA^{Short}) encoding a second cargo, wherein the first cargo and second cargo are not the same, wherein the first gRNA and second gRNA are not covalently linked, and wherein the payload flanked by the 5′ and 3′ LTRs of the first gRNA can be 3 times larger, measured in kilobase pairs, than the payload of the second gRNA. In some embodiments, the payload flanked by the 5′ and 3′ LTRs of the first gRNA can be at least 4 times larger than the payload of the second gRNA. In some embodiments, the payload flanked by the 5′ and 3′ LTRs of the first gRNA can be 4 times larger than the payload of the second gRNA. In some embodiments, the payload flanked by the 5′ and 3′ LTRs of the first gRNA can be at least 5 times larger than the payload of the second gRNA. In some embodiments, the payload flanked by the 5′ and 3′ LTRs of the first gRNA can be 5 times larger than the payload of the second gRNA. In some embodiments, the first gRNA can comprise a payload greater than 14 kb in size. In some embodiments, the combined payload of the first and second gRNAs can be less than 18 kb. In some embodiments, the first gRNA and second gRNA can comprise sequences that reduce or de-stabilize homopairing and/or increase or promote heteropairing.

In some embodiments, described herein are lentiviral vectors comprising a genome consisting essentially of one single RNA molecule (RNA^{single}). In some embodiments, the RNA^{single} can be greater than 10 kb in size. In some embodiments, the RVA^{single} can be greater than 12 kb-18 kb. In some embodiments, lentivirus vector particles can comprise an upstream packaging signal (PACK 1) and a downstream packaging signal (PACK 2), that associate, wherein PACK 1 and PACK 2 associate to form a structure recognized by a lentivirus dimerization-dependent packaging mechanism. In some embodiments, the PACK 1 can be positioned in the 5′ LTR. In some embodiments, the PACK 1 can be positioned downstream from the 5′ LTR. In some embodiments, the PACK 2 can be positioned in the 3′ LTR. In some embodiments, the PACK 2 can be positioned upstream from the 3′ LTR.

In another aspect, described herein are lentiviral vector particles capable of transducing a cell. In certain aspects, lentiviral vector particles as described herein can comprise a first gRNA encoding a first cargo and a second gRNA encoding a second cargo, wherein the first cargo and second cargo are not the same, wherein the first gRNA and second gRNA are not covalently linked, and wherein the first gRNA and second gRNA comprise sequences that reduce or de-stabilize homopairing. In some embodiments, the first gRNA and second gRNA can comprise sequences that increase or promote heteropairing. In some embodiments, the first gRNA can comprise an extended duplex sequence (EDS), wherein the EDS can comprise sequences corresponding to the dimer initiation signal (DIS) DIS, stem loop packaging sequence, splice donor (“SD”) and the AUG domains of HIV-1. In some embodiments, the second gRNA can comprise an extended duplex sequence (EDS), wherein the EDS can comprise one or more sequences corresponding to the DIS, stem loop packaging sequence, splice donor (“SD”) and AUG domains of HIV-1. In some embodiments, the first gRNA and the second gRNA can comprise stem-loop structures, and the stem structure adjacent to a dimer initiation signal (DIS) of the first gRNA and the second gRNA can be incompatible. In some embodiments, the stem-loop structures adjacent to the DIS can comprise sequences that reduce the thermodynamic favorability of homopairing and increase the favorability of heteropairing relative to corresponding sequences in native HIV-1. In some embodiments, the dimer initiation signal (DIS) of the first gRNA and/or the second gRNA can be nonpalindromic sequences. In some embodiments, the first gRNA and/or the second gRNA, the SD domain may not bind with an H1 domain and/or the gag AUG initiation codon (AUG) domain may not bind with U5. In some embodiments, 5′LTR of the first gRNA and/or the 5′LTR of the second gRNA can be modified relative to the HIV-1 genome [SEQ ID NO: 1] to increase the rate of heteropairing. In some embodiments, there can be a trans-activation responsive hairpin (TAR) hairpin, and the TAR of the first gRNA and/or second gRNA can be mutated or truncated relative to the TAR of the HIV-1 genome [SEQ ID NO:1]. In some embodiments, the 5′ LTR of either or both gRNAs can begin at the position corresponding to Position 2 of the HIV-1 gRNA sequence such that the cytosine base at Position 57 cannot be incorporated into the TAR hairpin stem.

In some embodiments, described herein are lentivirus vector particles comprising a first gRNA (gRNA^{Long}) encoding a first cargo, the first cargo encoding at least one non-viral polymer being a non-viral polypeptide or a non-viral polynucleotide, optionally a bacterial polypeptide or polynucleotide, a eukaryotic polypeptide or polynucleotide, an animal polypeptide or polynucleotide, a plant polypeptide or polynucleotide, a mammalian polypeptide or polynucleotide, a human polypeptide or polynucleotide, an antibody, an inhibitory RNA; or a guide RNA, or combination thereof, and a second gRNA (gRNA^{Short}), wherein the first gRNA and second gRNA are not covalently linked, and wherein the second gRNA: does not encode a polymer selected from a bacterial polypeptide and a bacterial polynucleotide; and/or does not encode a polymer selected from a eukaryotic polypeptide and a eukaryotic polynucleotide; and/or does not encode a polymer selected from an animal polypeptide and an animal polynucleotide; and/or does not encode a polymer selected from a plant polypeptide and a plant polynucleotide; and/or does not encode a polymer selected from a mammalian polypeptide and a mammalian polynucleotide; and/or does not encode a polymer selected from a human polypeptide and a human polynucleotide; and/or does not encode an antibody; and/or does not encode an inhibitory RNA; and/or does not encode a guide RNA. In some embodiments, the second gRNA may not encode a polypeptide that is not a viral polypeptide. In some embodiments, the second gRNA may not encode a protein other than one or a combination of the following: VSVG, Gag, Rev, Tat, Vpr, Vpx, Vif and Nef. In some embodiments, the second gRNA may not encode a protein. In some embodiments, the first gRNA can comprise a payload at least 3 times larger, measured in kilobase pairs, than the payload of the second gRNA. In some embodiments, the payload of the first gRNA can be at least 4 times larger than the payload of the second gRNA. In some embodiments, the first gRNA can comprise a payload 3 times larger, measured in kilobase pairs, than the payload of the second gRNA. In some embodiments, the payload of the first gRNA can be 4 times larger than the payload of the second gRNA. In some embodiments, the payload flanked by the 5′ and 3′ LTRs of the first gRNA can be at least 5 times larger than the payload of the second gRNA. In some embodiments, the payload flanked by the 5′ and 3′ LTRs of the first gRNA can be 5 times larger than the payload of the second gRNA. In some embodiments, the first gRNA can comprise a payload at least 14 kb or more in size. In some embodiments, the combined payload of the first and second gRNAs can be less than 18 kb. In some embodiments, the first gRNA and second gRNA can comprise sequences that reduce or de-stabilize homopairing and increase or promote heteropairing.

In some embodiments, described herein are lentivirus vector particles comprising a first gRNA (gRNA^{Long}) encoding a first cargo, the first cargo encoding at least one non-lentiviral polymer selected from a non-lentiviral polypeptide and a non-lentiviral polynucleotide, and a second gRNA (gRNA^{Short}) encoding a second cargo, wherein the first cargo and second cargo are not the same, wherein the first gRNA and second gRNA are not covalently linked, and wherein the payload flanked by the 5′ and 3′ LTRs of the first gRNA can be at least 3 times larger, measured in kilobase pairs, than the payload of the second gRNA. In some embodiments, described herein are lentivirus vector particles comprising a first gRNA (gRNA^{Long}) encoding a first cargo, the first cargo encoding at least one non-lentiviral polymer selected from a non-lentiviral polypeptide and a non-lentiviral polynucleotide, and a second gRNA (gRNA^{Short}) encoding a second cargo, wherein the first cargo and second cargo are not the same, wherein the first gRNA and second gRNA are not covalently linked, and wherein the payload flanked by the 5′ and 3′ LTRs of the first gRNA can be 3 times larger, measured in kilobase pairs, than the payload of the second gRNA. In some embodiments, the payload flanked by the 5′ and 3′ LTRs of the first gRNA can be at least 4 times larger than the payload of the second gRNA. In some embodiments, the payload flanked by the 5′ and 3′ LTRs of the first gRNA can be 4 times larger than the payload of the second gRNA. In some embodiments, the payload flanked by the 5′ and 3′ LTRs of the first gRNA can be at least 5 times larger than the payload of the second gRNA. In some embodiments, the payload flanked by the 5′ and 3′ LTRs of the first gRNA can be 5 times larger than the payload of the second gRNA. In some embodiments, the first gRNA can comprise a payload greater than 14 kb in size. In some embodiments, the combined payload of the first and second gRNAs can be less than 18 kb. In some embodiments, the first gRNA and second gRNA can comprise sequences that reduce or de-stabilize homopairing and/or increase or promote heteropairing.

In some embodiments, described herein are lentivirus vector particles comprising a genome consisting essentially of one single RNA molecule (RNA^{single}). In some embodiments, the RNA^{single} can be greater than 10 kb in size. In some embodiments, the RNA^{single} can be greater than 12 kb-18 kb. In some embodiments, lentivirus vector particles can comprise an upstream packaging signal (PACK 1) and a downstream packaging signal (PACK 2), that associate, wherein PACK 1 and PACK 2 associate to form a structure recognized by a lentivirus dimerization-dependent packaging mechanism. In some embodiments, the PACK 1 can be positioned in the 5′ LTR. In some embodiments, the PACK 1 can be positioned downstream from the 5′ LTR. In some embodiments, the PACK 2 can be positioned in the 3′ LTR. In some embodiments, the PACK 2 can be positioned upstream from the 3′ LTR.

Also described herein are kits comprising lentiviral vectors and lentiviral vector particles according to the present disclosure and instructions for use, as well as methods of using such.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows not-to-scale schematics illustrative of 2^{nd} and 3^{rd} generation transfer plasmid.

FIG. 2 illustrates, with reference to the 3^{rd} Generation Design plasmid as shown in FIG. 1, exemplary regions that can be modified. FIG. 2 shows RNA elements and does not include certain plasmid elements such as a promoter (e.g., CMV promoter).

FIG. 3 illustrates an embodiment of a stem loop sequence according to the present disclosure, given as 5′-CUCGGCUUGCUGAAGYGCRCWCRGCAAGAG-3′ [SEQ ID NO.2].

FIGS. 4A-4B illustrate embodiments of suitable modifications according to the present disclosure. FIG. 4A (taken from FIG. 6 of Dubois, supra) shows a schematic of a secondary structure model of the 5′-end region of the HIV-1 gRNA. In this illustration, a kissing-loop structure formed by two HIV-1 gRNA molecules is shown (top) and the extended duplex conformation is shown (bottom). FIG. 4B shows a similar image except that the sequence in stem region of each of the two gRNAs is modified to promote heterodimerization (“Hetero-pair A-B”) and reduce or destabilize homodimerization (“Homo-pair A-A” and “Homopair B-B”) by destabilizing the extended duplex conformation. An embodiment of a packaging sequence is located at “SL1-SL4” as shown in FIG. 4A. The sequence conservation and structure of a portion of the packaging sequence is also shown below (modified from https-en.wikipedia.org/wiki/Retroviral_psi_ packaging_element). Sequences are labeled and also reproduced in Section 7 below.

FIG. 5 is an illustration of embodiments of aspects of the present disclosure. As illustrated in FIG. 5, two transfer plasmids (T¹ and T²) each encoding a gRNA (gRNA¹ and gRNA²) are introduced into a cell. Each gRNA includes a 5′ UTR. Each of the two transfer plasmids includes a unique DNA sequence (UD¹ and UD²) inserted between the transcription +1 site and the first base of the 5′ R element of the respective gRNA. For illustration, T¹ encodes gRNA¹, which include UD¹, and T2 encodes gRNA², which includes UD². An identical UD sequence is then inserted into the opposite gRNA molecule between end of the PPT element and the 3′ R element. (i.e., the T¹ UD¹ sequence is added the T² 3′LTR, and the T² UD² sequence is added the T¹ 3′LTR).

FIG. 6 shows embodiments of exemplary structures of a long gRNA and several short gRNAs for use in a LENTI2-Y approach as described herein.

FIG. 7 shows a schematic for a long gRNA for use in a LENTI2-A approach as described herein. The location of the PACK2 at three different downstream locations 3′ to the 5′ LTR is indicated by black arrows.

FIG. 8 illustrates an embodiment of placement of PACK2 at three different downstream locations 3′ to the 5′ LTR.

FIG. 9 illustrates embodiments of Examples of PACK2 structures at the 3′ LTR according to the present disclosure.

FIG. 10 illustrates embodiments of additional possible positions of PACK2 as an alternative to being contained within the 3′ LTR.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Described herein are LVVs with a payload capacity that is roughly double that of the vectors currently in use, and methods for making and using such LVVs.

### 1. Abbreviations and Definitions

Unless otherwise defined, all terms of art, notations, and other scientific or medical terms or terminology used herein are intended to have the meanings commonly understood by those of ordinary skill in the art. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e., at least one) of the grammatical object of the article. By way of example, “an element” means at least one element and can include more than one element.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of those certain elements.” As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the present disclosure or features of the claims. See, for example, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.”Numerical quantities given herein are approximate unless stated otherwise, meaning that the term “about” or “approximately” can be inferred when not expressly stated. When “about” is applied to the beginning of a numerical range, it applies to both ends of the range.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, CRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double-and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful lentiviral vector may be used in the methods described herein. Examples for viral vectors include, lentiviral viral vectors. In some embodiments, the nucleic acid molecules are introduced into a cell using a lentiviral vector following standard procedures well known in the art, for example, by lentiviral transduction after the packing and formation of lentivirus particles which can then infect a host cell, introducing polynucleotides into the cell that had been packaged in the virus. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision.

The term “plasmid” refers to a nucleic acid molecule (i.e., a polynucleotide) that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

The term “vector” refers to a carrier DNA molecule (i.e., a polynucleotide) into which a DNA sequence can be inserted for introduction into a host cell. In some embodiments, vectors of use according to the present disclosure are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector, e.g., a promoter. Vectors include non-viral vectors such as plasmids and viral vectors, although, embodiments of lentiviral vectors in particular are provided for by the present disclosure.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include lentiviral vectors.

The term “operably linked” refers to a functional linkage between a first nucleic acid sequence and a second nucleic acid sequence, such that the first and second nucleic acid sequences are transcribed into a single nucleic acid sequence. Operably linked nucleic acid sequences need not be physically adjacent to each other. The term “operably linked” also refers to a functional linkage between a nucleic acid expression control sequence (such as a promoter, or array of transcription factor binding sites) and a transcribable nucleic acid sequence, wherein the expression control sequence directs transcription of the nucleic acid corresponding to the transcribable sequence.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances, the promoter sequence is recognized by the synthetic machinery of the cell, or introduced synthetic machinery, required for initiating transcription of a specific gene.

“Expression cassette” refers to a polynucleotide comprising a promoter or other regulatory sequence operably linked to a sequence encoding a protein.

The term “siRNA” refers to a nucleic acid that forms a double stranded RNA, which double stranded RNA has the ability to reduce or inhibit expression of a gene or target gene when the siRNA expressed in the same cell as the gene or target gene. In the context of the present disclosure, the term “siRNA” includes miRNA. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

The term “shRNA” refers generally to an siRNA that is introduced into a cell as part of a larger DNA construct. Typically, such constructs allow stable expression of the siRNA in cells after introduction, e.g., by integration of the construct into the host genome.

An “antisense” oligonucleotide or polynucleotide is a nucleotide sequence that is substantially complementary to a target polynucleotide or a portion thereof and has the ability to specifically hybridize to the target polynucleotide.

Ribozymes are enzymatic RNA molecules capable of catalyzing specific cleavage of RNA. The composition of ribozyme molecules preferably includes one or more sequences complementary to a target mRNA, and the well-known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “a recombination of amino acid sequences,” in the context of a peptide, refers to a change or variation in the amino acid sequence of a reference peptide, such that the biological properties of the reference peptide are maintained after the amino acid sequence change. For example, the recombination of amino acid sequence may be a conservative amino acid substitution or an amino acid sequence modification (addition, deletion or substitution) to produce a chimeric peptide.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4):581-87 (2013); Xie et al. “Adding amino acids to the genetic repertoire,” Curr. Opin. Chem. Biol. 9(6):548-54 (2005); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-10 and Altschul et al. (1977) Nucleic Acids Res. 25: 3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The term “library” is used according to its common usage in the art, to denote a collection of molecules, optionally organized and/or cataloged in such a way that individual members can be identified. Libraries can include, but are not limited to, combinatorial chemical libraries, natural products libraries, and peptide libraries.

The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/or differentiation such that the original cell is no longer exists, but the continuing cells will be understood to derive from the same source.

“Lentivirus” has the meaning known in the art of virology. Lentiviruses contain a dimeric genome of identical positive RNA strands complexed with nucleocapsid (or, equivalently, “capsid”) proteins.

“Lentivirus vector” or “LVV” is a lentivirus-derived viral vector generally comprising a lentivirus (or lentivirus-like) viral capsid containing RNA-genetic material, or other viral vector that contributes to the formation of functional lentivirus particles capable of transducing a cell and introducing genetic information or other genetic or polynucleotide payload into a cell. The LVV is capable of transducing the genetic material into mammalian host cells LVVs according to the present disclosure can also be packaged into functional lentiviruses, also referred to “lentivirus vector particles,” “viral particles,” “transducing particles,” or “virions.” See Munis, A. M., 2020, “Gene therapy applications of non-human lentiviral vectors,” Viruses 12.10:1106; Johnson et al., 2021, “HIV-based lentiviral vectors: origin and sequence differences,” Molecular Therapy-Methods & Clinical Development 21:451-465; Sertkaya et al., 2021, “HIV-1 sequences in lentiviral vector genomes can be substantially reduced without compromising transduction efficiency,” Sci Rep 11:12067, each of which is incorporated by reference. Such functional lentiviruses and lentiviral particles (comprising lentiviral vectors as described herein) according to the present disclosure are capable of transducing a host cell and introducing a genetic payload (packaged in the virus) into the host cell.

“LVV payload” refers to the genetic material contained in the portion of a gRNA between the LTRs of an LVV genomic RNA or to the combined portions from two gRNAs in a heterodimeric complex, as will be clear from context.

“LVV cargo” refers to means the portion of the LVV genetic material that encodes non-viral polypeptide(s) or polynucleotide(s) of interest. The non-viral polypeptides (e.g., interferon, CAS, PDGF) and polynucleotide(s)) are produced in an LVV-infected cell.

“gRNA” (genomic RNA) refers to a single RNA molecule contained in the capsid of an LVV as described hereinbelow. An LVV capsid generally contains two gRNAs, analogous to the dimeric genome of lentiviruses.

“Homozygous” in the context of an LVV means the LVV contains two identical or substantially identical gRNA molecules.

“Homopairing” (or equivalently “self-pairing”) refers to a noncovalent association of two gRNAs that are identical or substantially identical gRNA molecules. As will be clear from context, “homopairing” can also refer to the association of corresponding regions of two gRNA molecules (e.g., DIS sequences) that contribute to homopairing of the two gRNA molecules.

“Heterozygous” in the context of an LVV means the LVV contains two substantially different gRNA molecules, such two gRNA molecules encoding structurally and functionally divergent polypeptides or polynucleotides.

“Heteropairing” (or as used throughout the present disclosure equivalently: “trans-pairing”, “heterozygous pairing”, or “heterologous pairing”) refers to a noncovalent association of two gRNAs that are not identical or substantially identical gRNA molecules. As will be clear from context, “heteropairing” can also refer to the association of corresponding regions of two gRNA molecules (e.g., DIS sequence) that contribute to heteropairing of two not identical or substantially identical gRNA molecules.

“DLS” means “dimer linkage structure,” which is sometimes called a “dimeric packaging signal.”

“DIS” means “dimerization initiation signal” or “dimer initiation signal.”

“EDS” means “extended duplex sequence” and refers to HIV-1 gRNA positions 105-344.

“PSI” or “Ψ” means packaging signal sequence or packaging signal.

“U5” refers to the 5′ untranslated region of a long terminal repeat (LTR).

“U3” refers to the 3′ untranslated region of a long terminal repeat (LTR).

“PBS” means “primer binding site.”

“RRE” means “Rev-Responsive Element.”

“PPT” means “polypurine track.”

“cPPT” means “central polypurine tract.”

“P(CMV)” refers to a CMV promoter.

Two gRNAs (e.g., two gRNAs in the same capsid) are “substantially different” when they differ in size and/or sequence. Typically, substantially different gRNAs differ in size (measured in kilobases) by at least 10%. Typically, substantially different gRNAs share less than 90% nucleotide sequence identity and/or encode different polypeptides or promotor sequences. A gRNA of an LVV may be substantially different from another gRNA of the LVV, or may be substantially different from a naturally occurring lentivirus genome.

Two gRNAs are “structurally distinct” when they are substantially different in size or sequence. The two gRNAs are “functionally distinct” when they encode different polypeptides or different RNA transcripts. Generally, the different polypeptides or RNAs have different activities (e.g., IFN-gamma has a different activity than IFN-alpha) or functions (e.g., an shRNA has a function that is differs from the function of a single guide RNA (sgRNA)). In one example, a gRNA that encodes only viral proteins is substantially different from a gRNA that encodes at least one non-viral protein, such as a human protein or artificial polypeptide.

As used herein, “antibody” includes domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains.

As used herein, “inhibitory RNA” means antisense RNA, shRNA, miRNA, and siRNA.

As used herein, “a guide RNA” refers to a guide RNA or single guide RNA that includes a Cas-binding domain and sequence that directs the CAS to a genomic target to be modified.

The terms “stem-loop” and “hairpin” are used interchangeably and have the standard meeting in the art. A stem-loop forms when two regions of the same RNA strand base-pair to form a double helix that ends in an unpaired loop. The base-paired (or “annealed”) portion is the “stem region” and the unpaired portion is the “loop region.” Within a stem region base-pairing can be complete (the two strands in the step are perfectly complementary) or less than perfectly complementary. The stem may be formed annealing of a first RNA sequence and a second RNA sequence that is the reverse complement of the first. Stem-loop structures are discussed in Bevilacqua, P. C. and Blose, J. M., 2008, “Structures, Kinetics, Thermodynamics, and Biological Functions of RNA Hairpins” Annual Review of Physical Chemistry 59:1, 79-103, incorporated herein by reference.

The HIV-1 sequence is found at GenBank: AF033819.3 and provided herein as SEQ ID NO.: 1. See Section 7, below. Features of the HIV-1 genome, with reference to SEQ ID NO:1, include: 1 . . . 96—“repeat; positions of RNA transcription, initialization and polyadenylation; Region: R”; regulatory 73 . . . 78—regulatory_class=“polyA_signal_sequence”; 97 . . . 181-5′UTR; 182 . . . 199—primer_binding; 336 . . . 1838—gag gene/CDS; 1631 . . . 4642—pol gene/CDS; 4587 . . . 5165—vif gene/CDS; 5105 . . . 5341—vpr gene/CDS; 5377 . . . 7970—tat gene (CDS join: 5377 . . . 5591, 7925 . . . 7970); 5516 . . . 8199—rev gene (CDS join: 5516 . . . 5591, 7925 . . . 8199); 5608 . . . 5856—vpu gene/CDS; 5771 . . . 8341—env gene/CDS; 8343 . . . 8714—nef gene/CDS; 8631 . . . 9085-3′UTR; 9086 . . . 9181—“repeat; positions of RNA transcription initialization and polyadenylation; Region: R”. The mature polypeptides produced from the gag gene include: 339 . . . 731—matrix; 732 . . . 1424—capsid; 1425 . . . 1466—p2; 1467 . . . 1631—nucleocapsid; 1467 . . . 1631—p6 (coding regions noted). The mature polypeptides produced from the pol gene include: 1799 . . . 2095—protease; 2096 . . . 4174—reverse transcriptase; 4175 . . . 4639—integrase (coding regions noted). The env gene encodes a signal peptide (coding region 5771 . . . 5860). The mature polypeptides produced from the env gene include: 5861 . . . 7303—surface; 7304 . . . 8338—transmembrane envelope (coding regions noted).

This disclosure uses the phrase “corresponding to” to describe certain features of gRNAs. The genomic RNAs of LVVs described herein are derived, in part, from HIV-1 (see Section 7, below). The positions, sequences, and functions of features, domains, polypeptide and polynucleotide sequences in a gRNA disclosed herein can be referred to based on analogy to (i.e., sequence identity to) the HIV-1 reference sequence. The practitioner will be able to refer to an abundant literature and established nomenclature when referring to the structure, location and/or sequence of LTRs, dimer initiation signals, PSI sequences, stem-loop structures, and the like. In some instances, it may be useful to produce alignments (e.g., pairwise alignments) of an LVV gRNA with the HIV-1 reference sequence (SEQ ID NO:1). Such alignments can be carried out using art known means such as global alignment tools (e.g., EMBOSS Needle, EMBOSS Stretcher, GGEARCH2SEQ) which produce alignments based on the Needleman-Wunsch algorithm, and local alignment tools (e.g., EMBOSS Matcher, SSEARCH2) which produce alignments based on the Smith-Waterman algorithm. Pairwise alignments of similar sequences can also be prepared manually.

### 2. Introduction

Lentiviruses such as HIV are RNA viruses with a single-stranded positive sense RNA genome (gRNA). HIV, and LVVs derived from HIV, are ‘diploid’ in the sense that a viral particle contains two non-covalently associated copies of the RNA genome (gRNA) in a single capsid. It is believed that the diploid genome provides evolutionary advantages to lentiviruses, such as promoting genetic recombination, minimizing the effect of RNA damage by using processes such as template switching, and providing an identifiable dimer structure that allows viral encapsidation machinery to distinguish the viral genome from host mRNA. Like the lentiviruses from which they are derived, commonly used I. VVs are also diploid.

The diploid genomes of lentiviruses or LVVs arise from a non-covalent association (dimerization) of two RNAs. Lentiviral gRNA dimerization occurs via “kissing interactions” between GC-rich palindromic stem loops of each RNA molecule. The interaction results in structural transitions and intermolecular base pairing that result in a dimer linkage structure (DLS) and expose single-stranded motifs, known as the “packaging signal sequence” at the 5′ end regions of the gRNAs. The packaging signal sequence is also referred to as “packaging signal,” or “psi (Ψ).” The packaging signal sequence is recognized by the zinc finger domain of the nucleocapsid (NC) binding site of a Gag polyprotein. See Kim et al., 2012, “The determination of importance of sequences neighboring the Psi sequence in lentiviral vector transduction and packaging efficiency,” PLoS One 7.11: e50148; Durand S and Cimarelli A., 2011, “The inside out of lentiviral vectors,” Viruses 3(2):132-159. doi:10.3390/v3020132, both incorporated herein by reference.

Although the wild-type lentivirus genome usually contains two identical or nearly identical gRNAs, a cell that expresses two distinct dimer-compatible gRNAs may produce virions that contain two different gRNAs (i.e., gRNA heterodimers). See Johnson and Telesnitsky, 2010, “Retroviral RNA Dimerization and Packaging: The What, How, When, Where, and Why,” PLoS Pathog 6(10): e1001007 (doi.org/10.1371/journal.ppat.1001007). Also see Ali et al., 2016, “Cross-and Co-Packaging of Retroviral RNAs and Their Consequences,” Viruses 8(10):276 (doi:10.3390/v8100276).

We have determined that improved LVVs with a payload capacity roughly double that of conventional LVVs can be produced using one or more strategies described below. Some strategies described below include LVV designs that promote (or, equivalently “increase” or “stabilize”) or inhibit (or, equivalently “reduce” or “destabilize”) inter-molecular or intra-molecular association of sequences in two RNA molecules. Put differently, these features may reduce the thermodynamic favorability of homopairing and/or increasing the hermodynamic favorability of heteropairing. Thus, LLVs may be designed so that interactions between like-gRNAs (that when associate form a homodimer) are stabilized and/or interactions between unlike-gRNAs (that when associated form a heterodimer) are destabilized.

In naturally occurring lentiviruses, stem-loop structures play a key role in dimerization and packaging. For example, stem-loop structures are found in DIS and PSI regions. Destabilization of stem-loop regions to bias the thermodynamics of dimerization towards a desired structure is a strategy used in some approaches described hereinbelow.

SECTION 3 describes strategies involving co-packaging of two distinct gRNA. The two gRNAs are typically, but not necessarily, of similar length (e.g., 6-9 kb). Each of the gRNAs encodes at least one component (e.g., polypeptide or polynucleotide) essential to the intended end-use application of the vector. These strategies may be broadly described as LENTI2-X strategies.

SECTION 4 describes co-packaging of two gRNA molecules of substantially unequal length to produce high capacity LVVs. In one implementation of this strategy, a longer gRNA encodes the components required for the intended end-use application of the vector (e.g., all cargo) and a shorter gRNA encodes elements that allow for the effective viral packaging of a heterozygous virions (e.g., a packaging signal) but do not contain cargo required for the intended end-use of the LVV. The sum of the lengths of the long and short gRNAs can be up to 100% or more of the RNA capacity of a wild-type lentivirus particle virion (i.e., 18.4 kb). These strategies may be broadly described as LENTI2-Y strategies.

SECTION 5 describes strategies that can be considered a hybrid of LENTI2-Y and LENTI2-X and can be referred to as LENTI2-XY. This approach involves co-packaging of two gRNA molecules of substantially unequal length in which both the longer and the shorter gRNAs carry cargo encoding one or more components required for the intended end-use application of the vector and in which the longer gRNA is at least 3 times the length (measured in kilobases) of the shorter gRNA (e.g., 12.1 kb vs 4 kb).

SECTION 6 describes strategies involving LVVs in which a single, long “haploid” gRNA with a length of up to ˜18.4 kb or more is packaged in a single capsid. The single gRNA molecule encodes two functional viral packaging signals. An exemplary packaging signal is Ψ^{CES}, a minimal RNA structure required for efficient packaging of RNA into the HIV-1 capsid. See Heng et al., 2012, “Identification of a minimal region of the HIV-1 5′-leader required for RNA dimerization, NC binding, and packaging,” J. Mol. Bio. 417.3:224-239, incorporated herein by reference.) The intramolecular association of the two packaging signals acts as a molecular mimic for the dimeric packaging signal or dimer linkage structure (DLS) normally formed between two separate, smaller gRNAs in a wild-type homozygous diploid particle. The monomolecular dimer-mimic acts as a substitute for the DLS found in lentiviruses and conventional LVVs in seeding capsid formation. These strategies may be broadly described as LENTI2-A strategies.

Importantly, as will be apparent to a person of ordinary skill in the art reading this disclosure, many elements described in each of Sections 3-6 can be combined with elements from another section. As an example, methods for destabilizing homopairing and increasing heterologous pairing (i.e., “heteropairing”) described below in Section 3 may be combined with the strategies described in Sections 5 and 6. The organization of the disclosure into separate sections is solely intended for clarity and is not intended to limit combinations of elements from multiple different sections.

The reader will appreciate that illustrations herein are schematic and not intended to be comprehensive. A variety of additional modifications (relative to the illustrations or to wild-type HIV-1) are contemplated, including but not limited to combining elements separately described in different embodiments disclosed herein. Likewise, the skilled reader will be aware that modifications can be made to HIV-1 sequences involved in virus dimerization and assembly without loss of function. See, for example and not for limitation, Heng et al., 2012, “Identification of a Minimal Region of the HIV-1 5′-Leader Required for RNA Dimerization, NC Binding, and Packaging” J. Mol. Biol. 417:224-239, incorporated herein by reference.

### 2.1 Compositions

As would be understood from the present disclosure, described herein are compositions comprising lentiviral vectors and lentiviral vector particles. Such compositions can comprise, for example, polynucleotides as described herein. Polynucleotides as described herein can comprise various elements as described herein, for example, elements relating to the lentiviral genome and proteins required to be expressed in order for proper and efficient packaging and formation of functional lentiviruses capable of transducing a cell. Polynucleotides as described herein can also comprise one or more genes of interest (GOI) that are selected by the user. Such GOIs can be polynucleotides encoding protein products which the user desires to be introduced into and expressed by a cell (for example, a mammalian cell), either in vitro, in vivo, ex vivo, or by other means.

### 2.2 Kits

Compositions as described herein can be part of a kit. Kits comprising lentiviral vectors and lentiviral particles as described herein may have compositions as described herein provided for in a frozen aqueous solution, or dry lyophilized preparation, for example, that can be reconstituted by an end user by methods as known in the art. Such a kit may comprise a carrier means being compartmentalized to receive in close confinement one or more container means such as vials, tubes, and the like. The kit may also have containers containing buffer(s) and instructions for use.

### 2.3 Methods of Use

Also described herein are methods of using lentiviral vectors, lentiviral vector particles, and lentiviruses according to the present disclosure. Methods of use can include, for example, administering a vector or virus comprising a polynucleotide as described herein to one or more cells, with the aim of introducing polynucleotides contained in the vector or virus into the cell (past the cell membrane). Cells on which methods according to the present disclosure can utilize can be mammalian cells. Methods according to the present disclosure can be utilized in capacities such as, without intending to be limiting, in vitro, in vivo, and ex vivo.

The vectors comprising polynucleotides as described herein can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, nucleoporation, lentiviral infection (i.e., transduction), dextran-mediated transfection, polybrene-mediated transfection, protoplast fusion, and direct microinjection.

### 3. LVVS With Increased Payload Capacity Generated By Copackaging (LENTI2-X)

As noted above, it has been observed that, if a cell contains two distinct lentiviral genomes (i.e., transcribed from two provirus sequences), virions that contain gRNA heterodimers (e.g., a dimer containing two genomic RNAs with non-identical sequences within the same viral capsid) may be produced. In one aspect of the invention, LVV particles containing heterodimers with genomes that encode functionally and structurally distinct products. For example, one gRNA may encode a therapeutic growth factor (e.g., VEGF) and the second gRNA may encode a nanobody.

In nature, heterodimers that may be produced typically contain gRNA monomers with minimal sequence differences. Co-packaging of different gRNAs as a result of native HIV recombination between the two gRNAs has been observed and measured. See, e.g., Schlub et al., 2010, “Accurately measuring recombination between closely related HIV-1 genomes.” PLoS computational biology 6.4: e1000766. However, in these cases both genomes generally encode for the same (viral) proteins. Lentiviruses with heterodimeric genomes have also been inadvertently produced as an unintended consequence of preparing libraries of lentiviruses. See Feldman et al. 2018, “Lentiviral co-packaging mitigates the effects of intermolecular recombination and multiple integrations in pooled genetic screens.” BioRxiv 262121.

In one aspect of the present invention, heterodimeric LVV can be produced using a variety of strategies. In one approach, two different transfer plasmids (T¹ and T²) encoding distinct gRNAs (gRNA¹ and gRNA², respectively) are introduced into cell culture. If desired, drug resistance, fluorescence sorting, LeAPS, or other selection methods may be used to select cells transfected with both T¹ and T² plasmids, rather than only one. See Dobson et al., 2021, Antigen identification and high-throughput interaction mapping by reprogramming viral entry,” bioRxiv 2021.09.18.460796.

Alternatively, two gRNA encoding sequences may be carried on a single transfer plasmid to ensure co-delivery of different gRNAs to producer cells. In these approaches, some LVV particles are produced that carry two different RNA genomes. The distinct RNA genomes may be engineered to deliver two different genetic cargo; thus, the delivery capacity of the LVV is doubled.

However, these copackaging approach can be inefficient because in a producer cell expressing both gRNA¹ and gRNA² monomers only about 50% of resulting LVV particles result from copackaging of two different gRNAs (“heterozygous copackaging”) and about 50% of LVV particles result from copackaging of the same gRNA (“homozygous copackaging”). This disclosure provides methods to increase the rate of heterozygous co-packaging. It will be understood that methods may be used in various combinations as well as individually.

3.1 Modifying Packaging Sequences of gRNAs to De-Stabilize Homopairing and Improve the Rate of Heterologous Pairing Within the Producer Cells

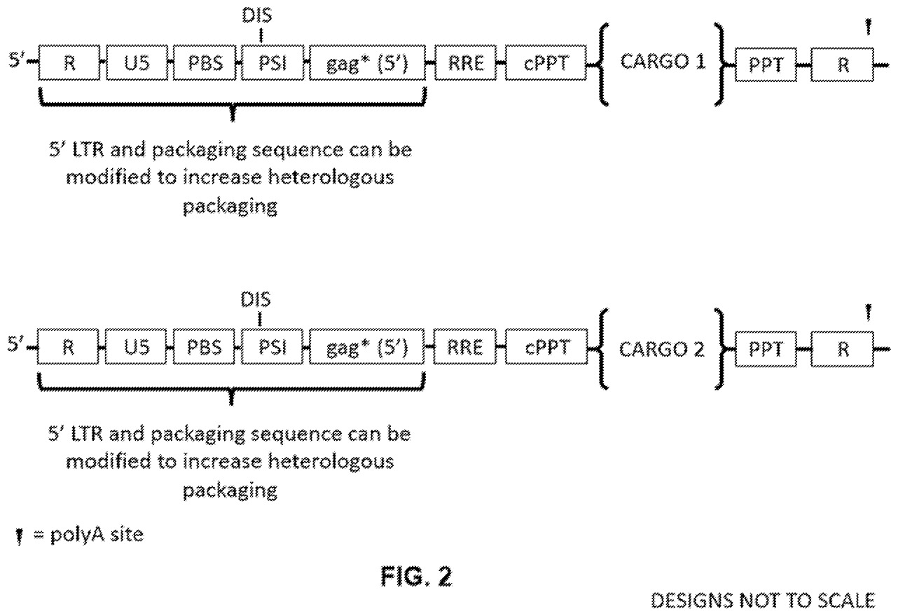

As noted above, specific sequences in the lentivirus genome play roles in dimerization. These sequences may be modified in an LVV to favor heteropairing (i.e., heterozygous pairing). FIG. 2 illustrates, with reference to the 3^{rd} Generation Design plasmid, exemplary regions that can be modified: FIG. 2 shows RNA elements and does not include certain plasmid elements such as a promoter (e.g., CMV promoter). A number of approaches, as described below, are contemplated to favor production of the dimeric (or extended) genomes of the lentivirus vectors of the invention.

3.1.1 Modifying a Stem Loop Structure Within the Packaging (PSI) Sequences of the gRNAs to Prevent Self-Pairing (Homopairing) and Promote Trans-Pairing.

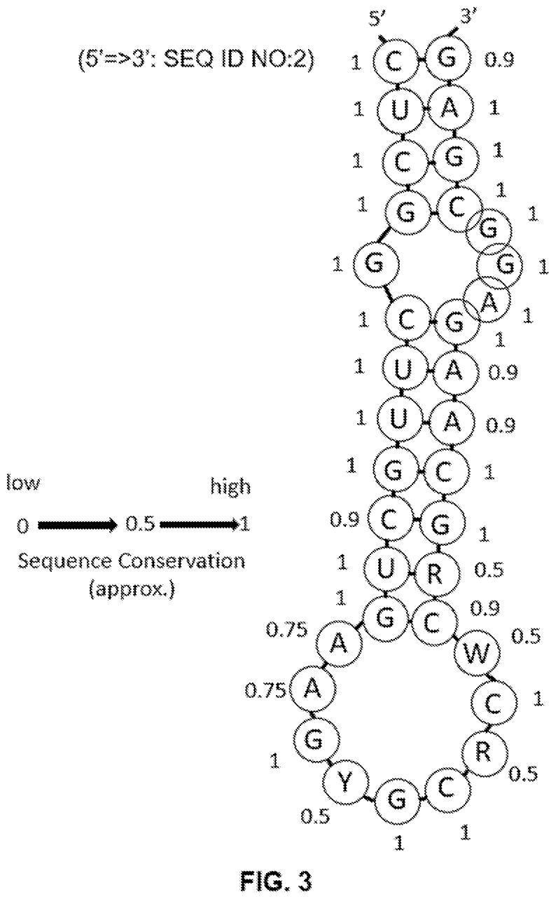

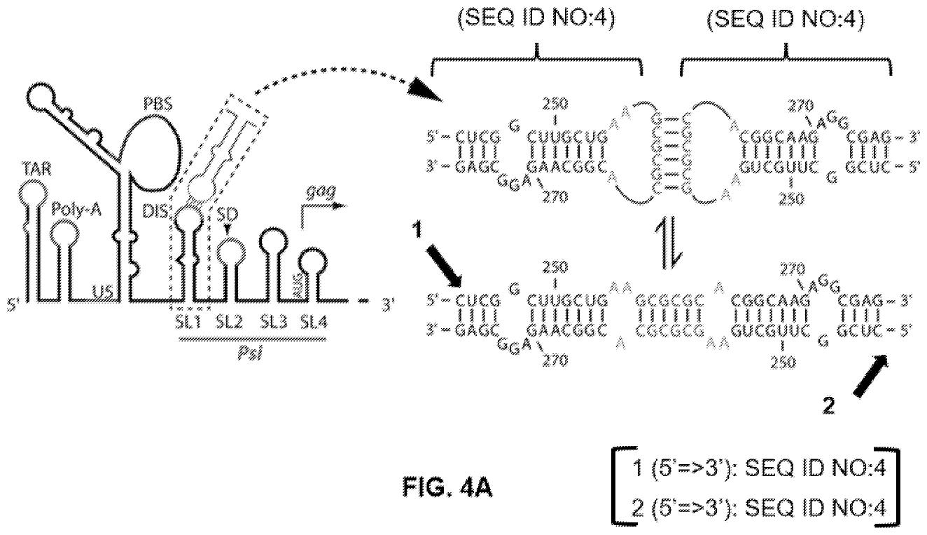

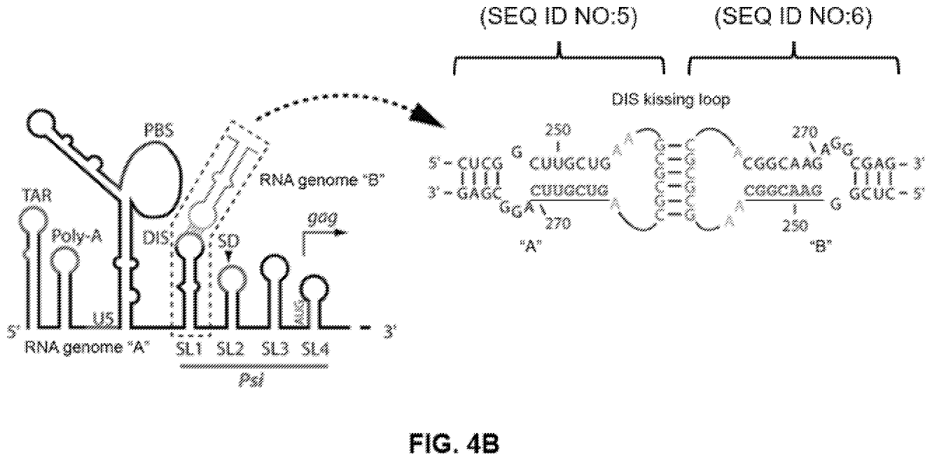

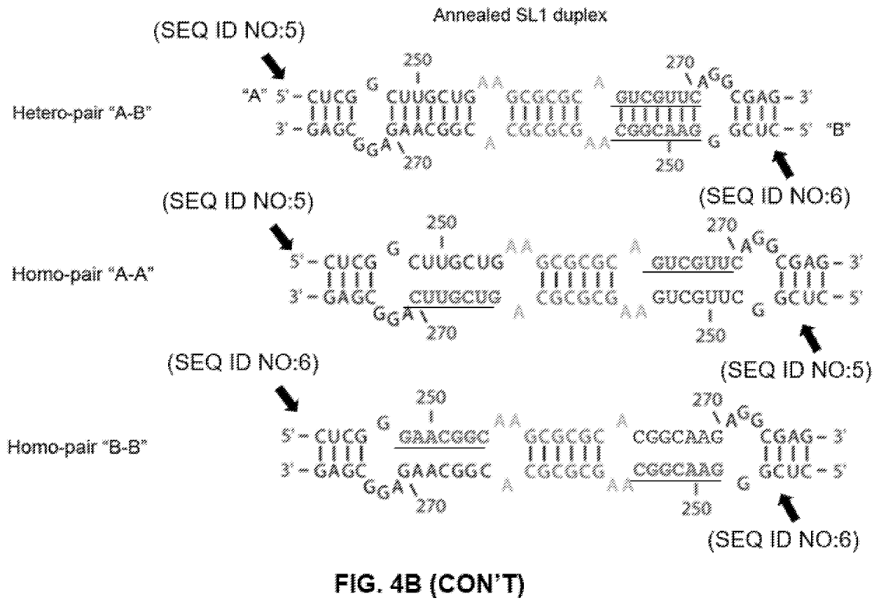

In one approach the stem loop structure is modified to reduce homopairing (or “self-pairing”) and promote heteropairing (or “heterologous pairing” or “transpairing”). Retroviral packaging sequences have been described by Dubois et al., 2018, “Retroviral RNA Dimerization: From Structure to Functions” Front. Microbiol., doi.org/10.3389/fmicb.2018.00527, incorporated herein by reference (hereinafter, “Dubois”). FIG. 4A, below, is a schematic drawing showing the packaging sequence (PSI) located at “SL1-SL4.” The sequence conservation and structure of a portion of the packaging sequence is also shown below (modified from https-en.wikipedia.org/wiki/Retroviral_psi_packaging_element). In FIG. 3, the stem loop sequence is given as 5′-CUCGGCUUGCUGAAGYGCRCWCRGCAAGAG-3′ [SEQ ID NO:2]. Y is C or T, R is G or A, and W is A. In one example, the sequence is 5′-CUCGGCUUGCUGAAGCGCGCACGGCAAGAG-3′ [SEQ ID NO:3]. Here, the canonical GCGCGC “DIS”, is located after the bottom most “AA”, and is shown by “GYGCRC” representing sequence variability at the 2^{nd} and 5^{th} positions. T.

In an approach the bases to the left and/or right of the DIS sequence in the “packaging sequence” are modified to deviate from the canonical sequence, so that self-dimerization is unfavored and trans-pairing is favored. Modifications may be selected to retain dimerization in the extended duplex within SL1 only for trans-pairing. That is, the SL1 stem sequences may be modified in a variety of ways so that the duplex is perfectly annealed in heterozygous pairs (bottom), but molecule cannot self-dimerize effectively. An example of this would be to flip some or all of the C-G, or A-U base pairs in the extended duplex. For example, in one approach, in the sequence to the right of the DIS (GCGCGCA), CGGCAAG is changed into GGCGUUC on the top molecule, and on the bottom molecule changes the bases to the right of the DIS from 5′-CUUGCUG-3′ into 5′-GAACGUC-3′.This design leaves the G-U mis-paired base unaffected, and switches all the fully complimentary sequences. In other embodiments fewer bases are switched. In some embodiments additional bases are switched (e.g. by adding new base pairs that trans-pair). Designs that switch fewer bases or more (adding new base pairs that trans-pair) also are options. The modification may be described as making the stem sequence 5′ to the DIS incompatible with stem sequence 3′ to the DIS. In another example, all the bases on one stem, except DIS, are modified to “A”, and all the bases on the other stem are modified to “T”. Both stems would be unable to self-bind, or self-dimerize, and would dimerize only in the presence of the other molecule. Any approach that disrupts self-binding of the stem, but compliments the changes to allow perfect binding with the other molecule may be used.

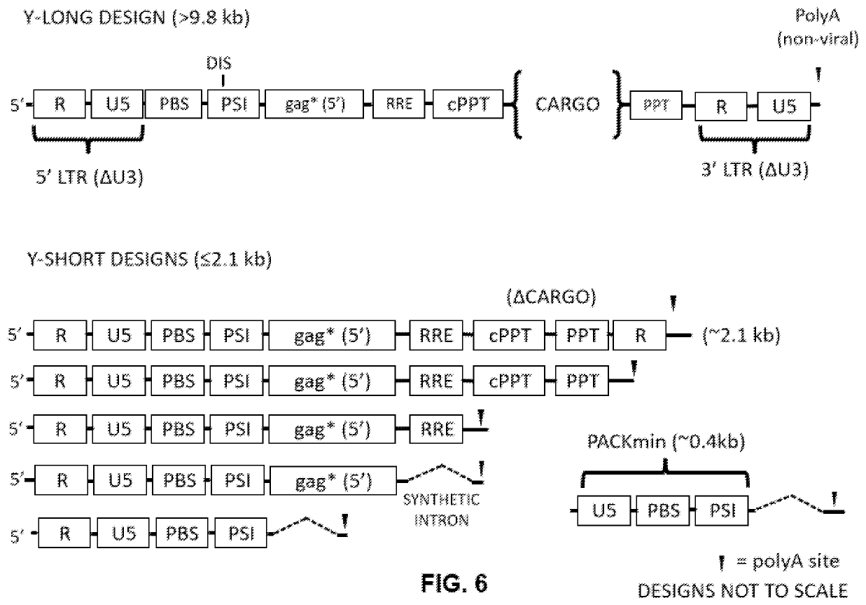

FIG. 4 illustrates an example of suitable modifications. FIG. 4A (taken from FIG. 6 of Dubois, supra) and shows a schematic secondary structure model of the 5′-end region of the HIV-1 gRNA. In this illustration a kissing-loop structure formed by two HIV-1 gRVA molecules is shown (top) and the extended duplex conformation is shown (bottom). FIG. 4B shows a similar image except that the sequence in stem region of each of the two gRNAs is modified to promote heterodimerization (“Hetero-pair A-B”) and reduce or destabilize homodimerization (“Homo-pair A-A” and “Homopair B-B”) by destabilizing the extended duplex conformation.

### 3.1.2 Changing the Dimer Initiation Signal (DIS) to Increase the Rate of Heterologous Genomic RNA Pairing Before Packaging

In an approach, the canonical DIS is “GYGCRC” (e.g., 5′-GCGCGC-′3) in each of the two gRNAs is replaced with non-palindromic DNA sequences, so self-pairing is disfavored. An example of a nonpalindromic sequence is GGGGGG (which pairs with CCCCCC). Other non-palindromic sequences are GGGGGC, GGCGGG, AGGGGG, and CGGGGGGC, which would be designed to pair with GCCCCC, CCCGCC, CCCCCT, and GCCCCCCG respectively. It will be appreciated that some non-palindromic pairs extend the number of bases in the DIS.

### 3.1.3 Changing Bases Within the Extended Duplex Sequence (EDS) Formed After Lentiviral Genome Pairing

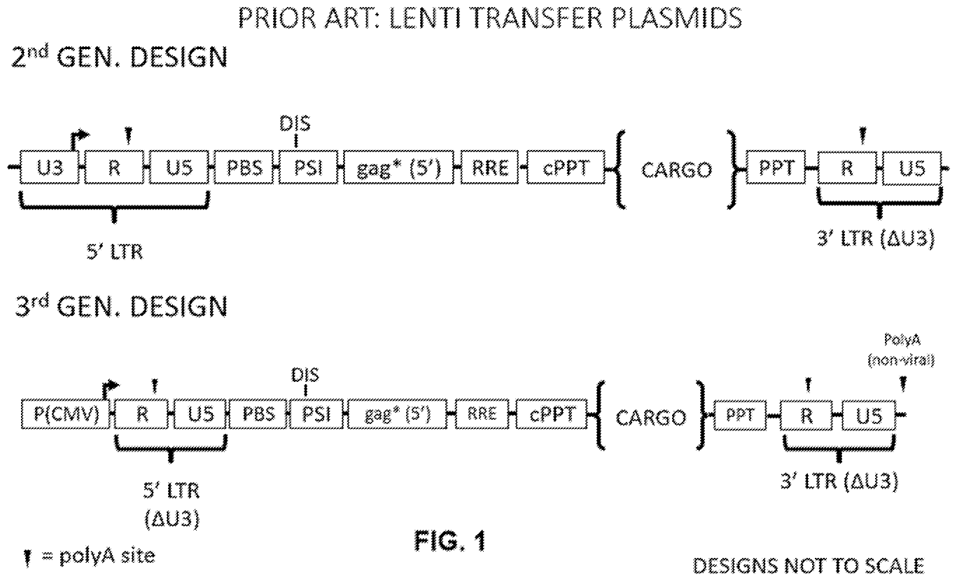

This approach extends the principle from Section 3.3.1 across the entire extended duplex sequence. As shown in FIG. 1A and 1B of Ding et al., 2020, “Identification of the initial nucleocapsid recognition element in the HIV-1 RNA packaging signal” PNAS 117:30, 17737-46, the extended duplex sequence includes “DIS” and the stem loop packaging sequence, but also extends through the Splice Donor (“SD”) and “AUG” domains of HIV. SD partially binds with H1 (a region of secondary structure after the DIS stem loop), and AUG binds with U5. See Ding et al. Modifications to these and other elements of the extended dimer sequence can also be used to produce heterodimeric LVVs. The bases can be modified as described in Section 3.1.1 such that self-dimerization is disfavored and trans-pairing is favored. An example of this includes modifying the AUG domain on one lentiviral genome “5′-AUGGG-3”, to “5′-UUCCC-3” (seen in Ding et al., FIG. 1A), and modifying the U5 domain on the second genome from “5′-CCCGU-3” to “5′-GGGGA-3′”. Self-dimerization will not be favored after extended duplex formation (Ding et al., FIG. 1B).

### 3.1.4 Changing Bases Within 5′ LTR, to Increase the Rate of Dimerization

### 3.1.4.1 Modifying the Trans-Activation Responsive (TAR) Element, the Polyadenylation Signal, or Both the TAR Element and the Polyadenylation Signal to Increase the Rate of Co-Pairing.

In one approach, bases within the 5′LTR are changed to increase the rate of heterozygous genome dimerization. Mutations in, or truncation of, the TAR hairpin may be used to increase the rate of HIV genome dimerization in different buffers. See Huthoff H and Berkhout B., 2001, Mutations in the TAR hairpin affect the equilibrium between alternative conformations of the HIV-1 leader RNA. Nucleic Acids Res. 29(12):2594-2600 (describing a 17 bp truncation of the TAR hairpin that increases genome dimerization compared to the WT sequence). The increase in the rate of dimerization may be used to compensate for other introduced features that are detrimental to virus formation to favor heterozygous genomic pairing but de-stabilize the dimer.

3.1.4.2 Removing the Initial GGG Sequence on the Lentiviral Genome and Replacing it by a Single G to Improve the Rate of gRNA Pairing.

Variation in the 5′ end of the WT HIV genome (through transcriptional start site heterogeneity) can affect the relative proportions of monomeric and dimerized genomes. Synthetic modifications to the 5′ end of the LVV genome can be used to increase the relative amount of dimeric genomes. In one approach, the promoters driving transfer plasmid RNA transcription are engineered to preferentially drive make only the equivalent of G₊₂ messages while avoiding the transcription of “wasteful” G₋₁ and G₊₁ messages. This strategy is based, in part, on the observation that in cells infected with HIV-1 viral RNAs containing a single 5′ capped guanosine (Cap1G) are specifically selected for packaging in virions. See Kharytonchyk et al., 2016, “Transcriptional start site heterogeneity modulates the structure and function of the HIV-1 genome,” Proc Natl Acad Sci USA. 2016 Nov. 22; 113 (47): 13378-13383 and Masuda et al., 2015, “Fate of HIV-1 cDNA intermediates during reverse transcription is dictated by transcription initiation site of virus genomic RNA,” Scientific Reports, 5:17680, both incorporated herein by reference. As reported, the native U3 promoter that drives gRNA expression in HIV-1 has some slippage with respect to its transcriptional start site. It can initiate transcription for any one of three downstream G residues at what corresponds to the 5′ LTR of the viral gRNA (G₋₁G₊₁G₊₂, by standard HIV-1 numbering). If the transcribed message starts at G₋₁ the transcript is preferentially spliced and used for protein synthesis and is not generally packaged into capsids (splicing deletes Psi from the transcript, rendering it packaging-incompetent). If transcription starts at G₊₂, the transcript remains unspliced (with Psi intact) and is preferentially packaged into capsids. If the transcript starts at G₊₁, it can be spliced or unspliced and used for either purpose, synthesis or packaging. As reported by Kharytonchyk et al., under physiological-like ionic conditions in which the Cap1G 5′-leader RNA adopts a dimeric structure, the Cap2G and Cap3G 5′-leader RNAs exist predominantly as monomers. Thus, it can be inferred that within the context of lentiviral vectors that a transfer plasmid's cargo transcripts that begin at G₋₁/G₊₁ are not as efficiently packaged as those that begin at G₊₂.

### 3.2 Functional Selection of Heterozygous Transducing Particles Via Obligatory Cross-Priming Reverse Transcription

In an approach, two distinct gRNAs are co-delivered into the same cell to produce heterozygous LVVs. Co-delivery may be affected by using two transfer plasmids (each encoding a different gRNA). The two gRNAs may be about the same length (e.g., ±1 kb) or may differ in length by two-fold or more.

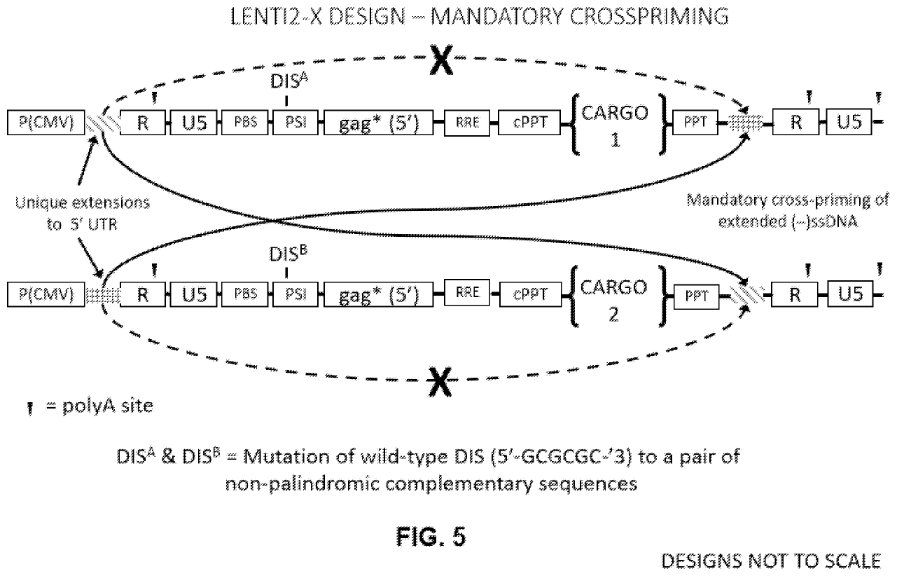

In this approach two transfer plasmids (T¹ and T²) each encoding a gRNA (gRNA¹ and gRNA²) are introduced into a cell. Each gRNA includes a 5′ UTR. Each of the two transfer plasmids includes a unique DNA sequence (UD¹ and UD²) inserted between the transcription +1 site and the first base of the 5′ R element of the respective gRNA. See FIG. 5. For illustration, T¹ encodes gRNA¹, which include UD¹, and T² encodes gRNA², which includes UD². An identical UD sequence is then inserted into the opposite gRNA molecule between end of the PPT element and the 3′ R element. (i.e., the T¹ UD¹ sequence is added the T² 3′LTR, and the T² UD² sequence is added the T¹ 3′LTR).

The added sequences ensure that the unique (-)ssDNAs originating from the original tRNA^{lys,3} primer extensions (reverse transcription) are incapable of self-priming minus-strand cDNA synthesis of their own respective gRNA molecule. Instead, the (-)ssDNA from T¹ can only prime further reverse transcription—on T², and vice versa. In such an arrangement, only heterozygous LVV particles can complete RT and transduce target cells. In contrast, homozygous LVV particles will be unable to prime reverse transcription effectively, and therefore will not efficiently transduce target cells.

### 4. High Capacity LVVs Generated By Copackaging a Long gRNA With Cargo and a Short gRNA Without Cargo (LENTI2-Y)

In another approach, LVVs with heterozygous genomes are produced by copackaging a long gRNA (gRNA^{Long}) with cargo and a minimal short gRNA (gRNA^{M-Short}) without cargo. In one implementation of this strategy, gRNA^{Long} contains sequences (i.e., cargo) encoding all the components required for the intended end-use application of the LVV, and gRNA^{M-Short} encodes elements required for gRNA dimerization, gRNA packaging and LVV capsid formation but does not contain cargo for the intended end-use of the LVV. In one approach the sum of the lengths of the long and short gRNAs are about 18 kb (the RNA capacity of a wild-type lentivirus particle virion) but the asymmetry in size allows the long gRNA to deliver sequences encoding therapeutic proteins that exceed the ˜9 kb limit of conventional homozygous LVVs. For example, in one approach the length of the gRNA^{M-Short} is about 4 kb, and the length of the gRNA^{Long} is 14_kb, long enough encode a large therapeutic protein (e.g., Dystrophin) as well as cis elements required for vector function (e.g., LTRs).

TABLE 1 illustrates relative sizes of a long and short gRNA based on the premise that the capacity of the long gRNA should be as great as possible (i.e., that the total length of the two gRNAs should be close to the maximum capacity of the lentivirus capsid. However, LVVs with a lower payload can be made when desired.

TABLE 1

| Length gRNA^{Long} (kb) | Length gRNA^{Short} (kb) |
|---|---|
| ~12-16 | ~2-4 |
| ~11-12 | ~5-6 |
| ~10-11 | ~7-8 |

In various embodiments the size (length in kb) of the long gRNA is at least 1.5-fold that of the short gRNA (e.g., ˜11 kb vs ˜7 kb), more often at least 2-fold the size of the short gRNA (e.g., ˜12 kb vs ˜6 kb), sometimes at least 3-fold the size of the short gRNA (e.g., ˜12 kb vs ˜4 kb), sometimes at least 3.5-fold the size of the short gRNA (e.g., ˜14 kb vs ˜4kb), sometimes at least 4-fold the size of the short gRNA (e.g., ˜16 kb vs ˜2 kb). In some embodiments the ratio of the size of the long gRNA to the short gRNA is at least about 1.5:1, 2:1, 2.5:1, 3:1, 3.5:1, 4:1, 5:1, 6:1, 7:1, or 8:1.

4.1 gRNA^{M-Short}

The cargo-less gRNA^{M-Short} can have a variety of elements or structures or features. In one approach the gRNA^{M-Short} has the structure of an “empty” transfer plasmid containing all of the native cis-elements, but lacking any inserted cargo. Using this approach, it will be apparent that any number of transfer plasmids described in the scientific and patent literature, some of which are commercially available, can be used in the LENTI2-Y by omitting any inserted cargo that would be included using conventional methods. The length of the native cis-elements is typically about 2.1 kb.

For illustration and not limitation, FIG. 6 shows exemplary structures of a long gRNA and several short gRNAs for use in a LENTI2-Y approach.

In one approach gRNA^{M-Short} contain a 5′ packaging sequence, but do not contain other components of a lentivirus genome.

In some cases, the gRNA^{M-Short} contain does not contain components for nuclear entry. For example, in one approach the 5′ and/or 3′ att sites are removed or inactivated, thereby preventing integrase binding and cDNA integration. The terminal 11 or 12 bases of the U5 and U3 att sites defines the minimum cis element required for integration or efficient interaction with HIV-1 integrase as described in Masuda et al., 1998, “Specific and independent recognition of U3 and U5 att sites by human immunodeficiency virus type 1 integrase in vivo,” J Virol. 72(10):8396-8402.

In some cases, the gRNA^{M-Short} does not contain a 3′-LTR.

In some cases, the gRNA^{M-Short} contains only a 5′ packaging sequence and a nuclear export sequence.

In some cases, the gRNA^{M-Short} contains only 5′ packaging sequence and an intron.

In some approaches the gRNA^{M-Short} encodes an RNA Pol-II driven transcript containing a minimal packaging signal (see Keane et al., 2015, RNA structure. Structure of the HIV-1 RNA packaging signal. Science. 2015;348(6237):917-921, FIG. 1A), a synthetic intron, and a polyadenylation signal. In this approach the RRE element is replaced with an intron. The intron is removed and the RNA is packaged as a smaller unit than it would be with RRE. For example, the post-spliced length may be in the range of 0.3-0.4 kb.

4.2 gRNA^{M-Long}

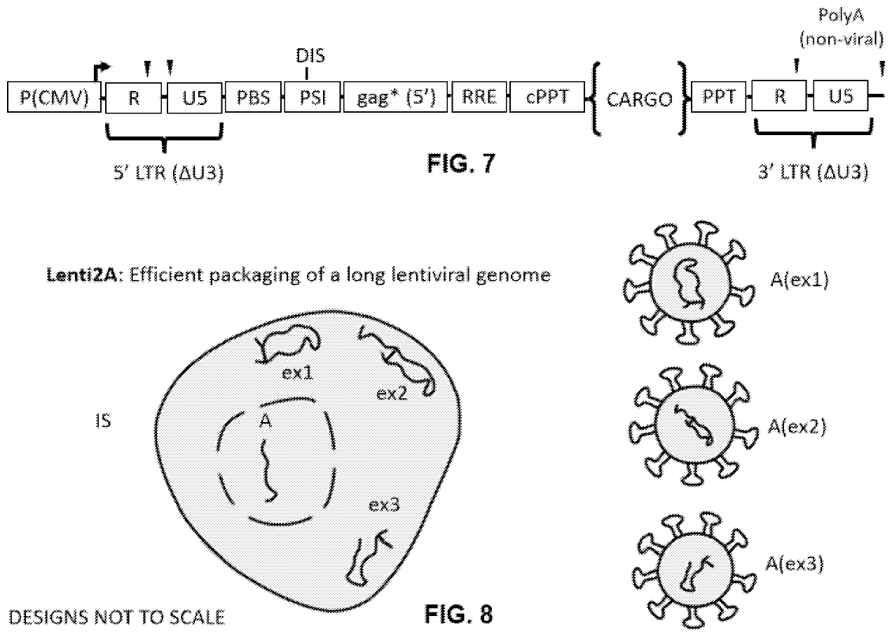

FIG. 7 shows an exemplary structure of a long gRNA for use in a LENTI2-Y approach. In one approach the gRNA^{Long} encodes wild-type versions of all the native viral cis-elements (totaling ˜2.1 kb) as well as up to ˜16-17 kb of cargo (e.g., sequences encoding one or more therapeutic polypeptides or polynucleotides). Using this approach, it will be apparent that any number of transfer plasmids described in the scientific and patent literature, some of which are commercially available, can be used in the LENTI2-Y method by inserting cargo that would be considered too long for use in conventional methods.

4.3 Modifying Packaging Sequences of gRNA^{M-Short} and gRNA^{Long} to De-Stabilize Homopairing and Improve the Rate of Heterologous Pairing Within The Producer Cells § 3.1

In § 3.1, above, methods are described for modifying gRNA sequences to favor production of heterologous LVVs. These methods may also be used in combination with other approaches, including the LENTI2-Y designs described in Sec. 2, above.

4.4 Homozygous gRNA Pairings Do Not Result in Productive Transducing Particles.

Advantageously, LENTI2-Y selects against production of homozygous LVVs.

gRNA^{Long} and gRNA^{Long} (“LONG-LONG”) homozygotes will exceed the 18.4 kb total RNA limit for the lentiviral capsid, and therefore will generally fail to package. Moreover, even if a ‘Long-Long’ LVV is produced, the resulting particle will still encode all of the synthetic components required for the end-use application of the vector, just now at a copy number of 2 instead of 1.

Packaging of gRNA^{M-Short} and gRNA^{M-Short} (“SHORT-SHORT”) homozygotes is not necessarily precluded by size, depending on the design. However, a SHORT-SHORT homozygote would not include at least one element required in the lentiviral life cycle, and would not form productive transducing particles.

### 5. High Capacity LVVS Generated By Copackaging a Long gRNA and a Short gRNA, Both With Cargo (LENTI2-XY)

In an approach similar to LENTI2-Y, LVVs containing an asymmetrically sized pair of gRNAs is used. TABLE 2 illustrates hypothetical size differences between gRNA^{Long} and gRNA_{Short}. In contrast to LENTI2-Y, in which gRNA^{M-Short} does not include cargo, gRNAS^{Short} contains sequences encoding small components such as nanobodies, shRNA and the like, so that the gRNA cargo sizes are also asymmetric. Table 1 shows exemplary size ranges, for illustration and not limitation.

TABLE 2

| Length gRNA^{Long} (KB) | Length gRNA^{Short} (KB) |
|---|---|
| ~12-16 | ~1-2 |
| ~11-12 | ~4-5 |
| ~10-11 | ~5-6 |

5.1 Examples of Polypeptides and Polynucleotides Contained in Long and Short gRNAs

TABLE 3 illustrate examples of cargo that can be encoded in gRNAs.

TABLE 3

| gRNA^{Long} (KB) | gRNA^{Short} (KB) |
|---|---|
| Prime Editor | pegRNA |
| Cas9 + long nucleic acid | gRNA |
| donor (<6 kb) | |
| Modified Cas editor (dCas9 + | gRNA |
| SSAP) + donor (<4 kb) | |
| Cas9 | Short nucleic acid “donor” |
| Therapeutic payload (Dystrophin) | shRNA (inhibiting disease gene) |
| Transgene expressed via | Transcription factor (TF) |
| TF-modulated promoter | |
| Genetic complement restoring | shRNA targeting endogenous mutant |
| wild-type function | allele |

*Scholefield, J., Harrison, P. T., 2021, “Prime editing - an update on the field” Gene Ther 28, 396-401.
**Wang et al., 2022, “dCas9-based gene editing for cleavage-free genomic knock-in of long sequences” (pp. 1-11). Nature Publishing Group.

### 6. Single gRNA (gRNA^{Single}) LVVs (LENTI-2A)

In one approach, high capacity LVVs are designed so that a single long gRNA (“gRNA^{Single}”) is packaged per LVV capsid. In one approach, the gRNA^{Single} contains two compatible packaging signal sequences (PSI). The intramolecular association of the two packaging signals will act as a molecular mimic (“DLS-mimic”) for the DLS complex normally formed between the two separate, smaller RNA cargo molecules found in a wild-type homozygous diploid particle. The monomolecular DLS-mimic would then act as a substitute for its native DLS in seeding capsid formation and continuing viral particle synthesis. Using this approach, a single, long gRNA (with a length of up to about ˜18.4 kb) molecule is packaged. In some embodiments the total length of the gRNA^{Single} is in the range of about 12 kb to 18 kb, or about 15 kb to 18 kb.

The DLS-mimic is formed from two sequences corresponding to two PSIs in WT lentivirus or conventional LVVs. The two packaging signal sequences are referred to herein as PACK1 (upstream sequence) and PACK2 (downstream sequence). The positions and orientations of PACK1 and PACK2 are selected to allow the sequences to associate (“self-pair) and form a DLS-mimic recognized by the lentivirus dimerization-dependent packaging mechanism.

In some approaches, PACK1 is placed near the 5′ UTR downstream from the R element (generally where it is positioned in commonly used transfer plasmids) and PACK2 is placed at a downstream, location. FIG. 8 illustrates placement of PACK2 at three different downstream locations 3′ to the 5′ LTR.

PACK1 and PACK2 typically have the same sequence, such as an HIV-1 PSI or other lentivirus sequence. Alternatively, PACK1 and PACK2 may have different sequences, provided they interact with each other to from a duplex recognized by the lentivirus dimerization-dependent packaging mechanism. An example of an assay that can be used to determine whether a candidate DLS-mimic is recognized by a lentivirus dimerization-dependent packaging mechanism is provided in Tran et al., 2015, “Conserved determinants of lentiviral genome dimerization,” Retrovirology 12, 83, FIG. 1.

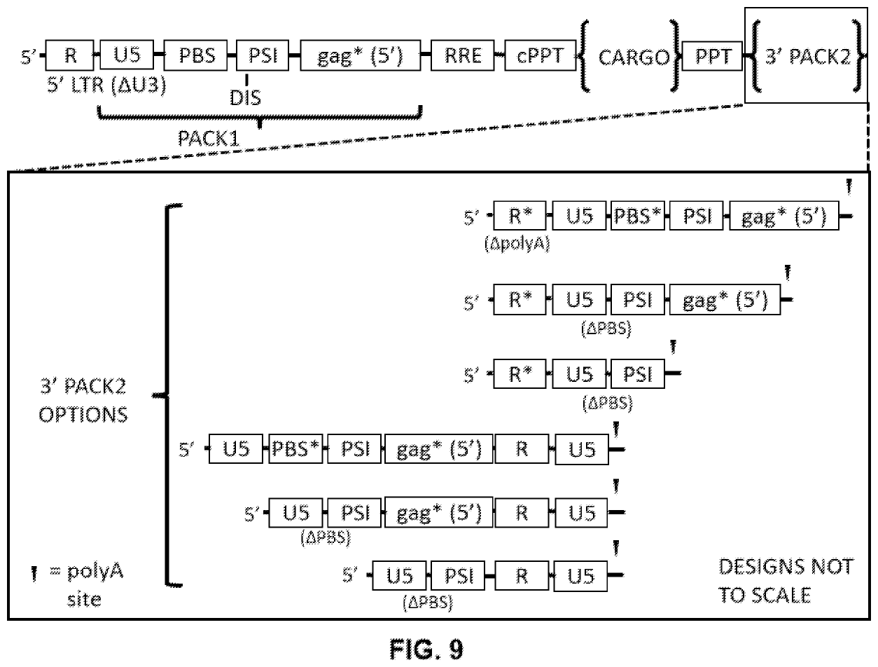

Each position of PACK1 (in FIG. 8) can be combined with PACK2 structures that enhance packaging and can be either retained or removed during reverse transcription. Examples of PACK2 structures are illustrated in FIG. 9. In one approach the 3′ packaging signal (PACK2) is contained in the 3′ LTR. This signal can include a variety of wild-type and mutant cis-elements in different combinations. Such elements include, but are not limited to, U5, the PBS, PSI (i.e., SL1-SL4 secondary structures), an alternative non-native polyadenylation signal, and/or the 5′ end of the GAG sequence, all of which may aid in efficient packaging. Possible mutations to wild-type cis elements include, but are not limited to, mutations to R to remove its native polyadenylation signal, deletion or truncation of the PBS loop (while leaving other stems and loops unperturbed to eliminate priming of reverse transcription with tRNA^{lys,3} and/or frameshift mutations in the GAG nucleotide sequence to abrogate the production of gag protein. The PACK2 cis-elements can be placed on either side of the 3′ LTR “R” element, where the reverse primer (-)sscDNA binds following first strand transfer of reverse transcription of the genome. Upon completion of the the lentivirus reverse-transcription cycle, the final cDNA sequence of all elements to the right of “R” are defined not by the 3′ end of the gRNA, but instead by the sequence of the incoming (-)sscDNA. Thus, gRNA elements 3′ to the 3′ LTR R segment are not reverse transcribed, and instead are deleted from the final DNA delivered to the cell while all elements to the left of “R” will be retained in the reverse transcribed (dsDNA) lentiviral genome.

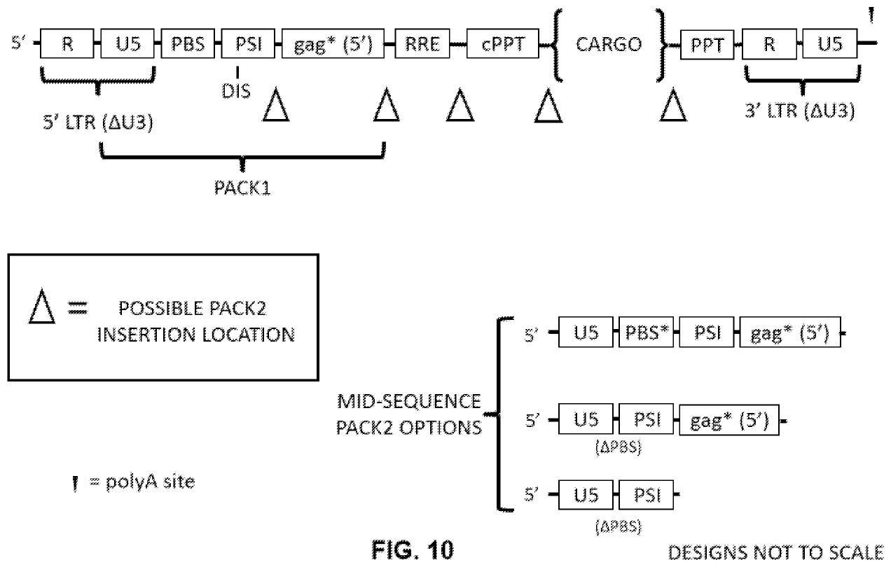

FIG. 10 illustrates additional possible positions of PACK2 as an alternative to being contained within the 3′ LTR. These alternative positions include: (i) between PSI and gag; (ii) between gag and RRE, (iii) 3′ to RRE, (iv) 5′ to cargo (between cPPT and cargo) and (v) 3′ to cargo (between cargo and PPT). In one approach the PACK2 neighbors the native original packaging signal, e.g., two packaging signals are next to each other and form a direct repeat, with <100 nucleotides separating them. This duplex packaging signal can self-bind even at close proximity.

PACK2 signals located at these alternative positions may be composed of a variety of wild-type and mutant cis-elements in different combinations and relative orientations. Such elements include, but are not limited to, U5, the PBS, PSI (i.e., SL1-SL4 secondary structures), and/or the 5′ end of the GAG nucleotide sequence. Possible mutations include, but are not limited to, those described above in this section.

Intramolecular pairing of PACK1 and PACK2 will be entropically favored over any intermolecular pairings, based on the principle of increased effective concentration of intramolecular ligands. In addition, strategies described in § 3.1, above, may be used to increase the probability of intramolecular PACK1/PACK2 association over intermolecular associations.

### 7. Sequences of the Present Disclosure: HIV-1 gRNA and Others

An annotated version of the HIV-1 gRNA is provided below. HIV-1 gRNA domains is described below. Also see Ding et al., 2020, “Identification of the initial nucleocapsid recognition element in the HIV-1 RNA packaging signal,” Biochemistry 117 (30) 17737-17746.

SEQ ID NO.: 1 GenBank: AF033819.3-HIV-1, complete genome, 9181 bp, linear RNA, ver. 21-Sep.-2018.

| | |
|---|---|
| 1 | ggtctctctg gttagaccag atctgagcct gggagctctc tggctaacta gggaacccac |
| 61 | tgcttaagcc tcaataaagc ttgccttgag tgcttcaagt agtgtgtgcc cgtctgttgt |
| 121 | gtgactctgg taactagaga tccctcagac ccttttagtc agtgtggaaa atctctagca |
| 181 | gtggcgcccg aacagggacc tgaaagcgaa agggaaacca gaggagctct ctcgacgcag |
| 241 | gactcggctt gctgaagcgc gcacggcaag aggcgagggg cggcgactgg tgagtacgcc |
| 301 | aaaaattttg actagcggag gctagaagga gagagatggg tgcgagagcg tcagtattaa |
| 361 | gcgggggaga attagatcga tgggaaaaaa ttcggttaag gccaggggga aagaaaaaat |
| 421 | ataaattaaa acatatagta tgggcaagca gggagctaga acgattcgca gttaatcctg |
| 481 | gcctgttaga aacatcagaa ggctgtagac aaatactggg acagctacaa ccatcccttc |
| 541 | agacaggatc agaagaactt agatcattat ataatacagt agcaaccctc tattgtgtgc |
| 601 | atcaaaggat agagataaaa gacaccaagg aagctttaga caagatagag gaagagcaaa |
| 661 | acaaaagtaa gaaaaaagca cagcaagcag cagctgacac aggacacagc aatcaggtca |
| 721 | gccaaaatta ccctatagtg cagaacatcc aggggcaaat ggtacatcag gccatatcac |
| 781 | ctagaacttt aaatgcatgg gtaaaagtag tagaagagaa ggctttcagc ccagaagtga |
| 841 | tacccatgtt ttcagcatta tcagaaggag ccaccccaca agatttaaac accatgctaa |
| 901 | acacagtggg gggacatcaa gcagccatgc aaatgttaaa agagaccatc aatgaggaag |
| 961 | ctgcagaatg ggatagagtg catccagtgc atgcagggcc tattgcacca ggccagatga |
| 1021 | gagaaccaag gggaagtgac atagcaggaa ctactagtac ccttcaggaa caaataggat |
| 1081 | ggatgacaaa taatccacct atcccagtag gagaaattta taaaagatgg ataatcctgg |
| 1141 | gattaaataa aatagtaaga atgtatagcc ctaccagcat tctggacata agacaaggac |
| 1201 | caaaggaacc ctttagagac tatgtagacc ggttctataa aactctaaga gccgagcaag |
| 1261 | cttcacagga ggtaaaaaat tggatgacag aaaccttgtt ggtccaaaat gcgaacccag |
| 1321 | attgtaagac tattttaaaa gcattgggac cagcggctac actagaagaa atgatgacag |
| 1381 | catgtcaggg agtaggagga cccggccata aggcaagagt tttggctgaa gcaatgagcc |
| 1441 | aagtaacaaa ttcagctacc ataatgatgc agagaggcaa ttttaggaac caaagaaaga |
| 1501 | ttgttaagtg tttcaattgt ggcaaagaag ggcacacagc cagaaattgc agggccccta |
| 1561 | ggaaaaaggg ctgttggaaa tgtggaaagg aaggacacca aatgaaagat tgtactgaga |
| 1621 | gacaggctaa ttttttaggg aagatctggc cttcctacaa gggaaggcca gggaattttc |
| 1681 | ttcagagcag accagagcca acagccccac cagaagagag cttcaggtct ggggtagaga |
| 1741 | caacaactcc ccctcagaag caggagccga tagacaagga actgtatcct ttaacttccc |
| 1801 | tcaggtcact ctttggcaac gacccctcgt cacaataaag ataggggggc aactaaagga |
| 1861 | agctctatta gatacaggag cagatgatac agtattagaa gaaatgagtt tgccaggaag |
| 1921 | atggaaacca aaaatgatag ggggaattgg aggttttatc aaagtaagac agtatgatca |
| 1981 | gatactcata gaaatctgtg gacataaagc tataggtaca gtattagtag gacctacacc |
| 2041 | tgtcaacata attggaagaa atctgttgac tcagattggt tgcactttaa attttcccat |
| 2101 | tagccctatt gagactgtac cagtaaaatt aaagccagga atggatggcc caaaagttaa |
| 2161 | acaatggcca ttgacagaag aaaaaataaa agcattagta gaaatttgta cagagatgga |
| 2221 | aaaggaaggg aaaatttcaa aaattgggcc tgaaaatcca tacaatactc cagtatttgc |
| 2281 | cataaagaaa aaagacagta ctaaatggag aaaattagta gatttcagag aacttaataa |
| 2341 | gagaactcaa gacttctggg aagttcaatt aggaatacca catcccgcag ggttaaaaaa |
| 2401 | gaaaaaatca gtaacagtac tggatgtggg tgatgcatat ttttcagttc ccttagatga |
| 2461 | agacttcagg aagtatactg catttaccat acctagtata aacaatgaga caccagggat |
| 2521 | tagatatcag tacaatgtgc ttccacaggg atggaaagga tcaccagcaa tattccaaag |
| 2581 | tagcatgaca aaaatcttag agccttttag aaaacaaaat ccagacatag ttatctatca |
| 2641 | atacatggat gatttgtatg taggatctga cttagaaata gggcagcata gaacaaaaat |
| 2701 | agaggagctg agacaacatc tgttgaggtg gggacttacc acaccagaca aaaaacatca |
| 2761 | gaaagaacct ccattccttt ggatgggtta tgaactccat cctgataaat ggacagtaca |
| 2821 | gcctatagtg ctgccagaaa aagacagctg gactgtcaat gacatacaga agttagtggg |
| 2881 | gaaattgaat tgggcaagtc agatttaccc agggattaaa gtaaggcaat tatgtaaact |
| 2941 | ccttagagga accaaagcac taacagaagt aataccacta acagaagaag cagagctaga |
| 3001 | actggcagaa aacagagaga ttctaaaaga accagtacat ggagtgtatt atgacccatc |
| 3061 | aaaagactta atagcagaaa tacagaagca ggggcaaggc caatggacat atcaaattta |
| 3121 | tcaagagcca tttaaaaatc tgaaaacagg aaaatatgca agaatgaggg gtgcccacac |
| 3181 | taatgatgta aaacaattaa cagaggcagt gcaaaaaata accacagaaa gcatagtaat |
| 3241 | atggggaaag actcctaaat ttaaactgcc catacaaaag gaaacatggg aaacatggtg |
| 3301 | gacagagtat tggcaagcca cctggattcc tgagtgggag tttgttaata cccctccctt |
| 3361 | agtgaaatta tggtaccagt tagagaaaga acccatagta ggagcagaaa ccttctatgt |
| 3421 | agatggggca gctaacaggg agactaaatt aggaaaagca ggatatgtta ctaatagagg |
| 3481 | aagacaaaaa gttgtcaccc taactgacac aacaaatcag aagactgagt tacaagcaat |
| 3541 | ttatctagct ttgcaggatt cgggattaga agtaaacata gtaacagact cacaatatgc |
| 3601 | attaggaatc attcaagcac aaccagatca aagtgaatca gagttagtca atcaaataat |
| 3661 | agagcagtta ataaaaaagg aaaaggtcta tctggcatgg gtaccagcac acaaaggaat |
| 3721 | tggaggaaat gaacaagtag ataaattagt cagtgctgga atcaggaaag tactattttt |
| 3781 | agatggaata gataaggccc aagatgaaca tgagaaatat cacagtaatt ggagagcaat |
| 3841 | ggctagtgat tttaacctgc cacctgtagt agcaaaagaa atagtagcca gctgtgataa |
| 3901 | atgtcagcta aaaggagaag ccatgcatgg acaagtagac tgtagtccag gaatatggca |
| 3961 | actagattgt acacatttag aaggaaaagt tatcctggta gcagttcatg tagccagtgg |
| 4021 | atatatagaa gcagaagtta ttccagcaga aacagggcag gaaacagcat attttctttt |
| 4081 | aaaattagca ggaagatggc cagtaaaaac aatacatact gacaatggca gcaatttcac |
| 4141 | cggtgctacg gttagggccg cctgttggtg ggcgggaatc aagcaggaat ttggaattcc |
| 4201 | ctacaatccc caaagtcaag gagtagtaga atctatgaat aaagaattaa agaaaattat |
| 4261 | aggacaggta agagatcagg ctgaacatct taagacagca gtacaaatgg cagtattcat |
| 4321 | ccacaatttt aaaagaaaag gggggattgg ggggtacagt gcaggggaaa gaatagtaga |
| 4381 | cataatagca acagacatac aaactaaaga attacaaaaa caaattacaa aaattcaaaa |
| 4441 | ttttcgggtt tattacaggg acagcagaaa tccactttgg aaaggaccag caaagctcct |
| 4501 | ctggaaaggt gaaggggcag tagtaataca agataatagt gacataaaag tagtgccaag |
| 4561 | aagaaaagca aagatcatta gggattatgg aaaacagatg gcaggtgatg attgtgtggc |
| 4621 | aagtagacag gatgaggatt agaacatgga aaagtttagt aaaacaccat atgtatgttt |
| 4681 | cagggaaagc taggggatgg ttttatagac atcactatga aagccctcat ccaagaataa |
| 4741 | gttcagaagt acacatccca ctaggggatg ctagattggt aataacaaca tattggggtc |
| 4801 | tgcatacagg agaaagagac tggcatttgg gtcagggagt ctccatagaa tggaggaaaa |
| 4861 | agagatatag cacacaagta gaccctgaac tagcagacca actaattcat ctgtattact |
| 4921 | ttgactgttt ttcagactct gctataagaa aggccttatt aggacacata gttagcccta |
| 4981 | ggtgtgaata tcaagcagga cataacaagg taggatctct acaatacttg gcactagcag |
| 5041 | cattaataac accaaaaaag ataaagccac ctttgcctag tgttacgaaa ctgacagagg |
| 5101 | atagatggaa caagccccag aagaccaagg gccacagagg gagccacaca atgaatggac |
| 5161 | actagagctt ttagaggagc ttaagaatga agctgttaga cattttccta ggatttggct |
| 5221 | ccatggctta gggcaacata tctatgaaac ttatggggat acttgggcag gagtggaagc |
| 5281 | cataataaga attctgcaac aactgctgtt tatccatttt cagaattggg tgtcgacata |
| 5341 | gcagaatagg cgttactcga cagaggagag caagaaatgg agccagtaga tcctagacta |
| 5401 | gagccctgga agcatccagg aagtcagcct aaaactgctt gtaccaattg ctattgtaaa |
| 5461 | aagtgttgct ttcattgcca agtttgtttc ataacaaaag ccttaggcat ctcctatggc |
| 5521 | aggaagaagc ggagacagcg acgaagagct catcagaaca gtcagactca tcaagcttct |
| 5581 | ctatcaaagc agtaagtagt acatgtaatg caacctatac caatagtagc aatagtagca |
| 5641 | ttagtagtag caataataat agcaatagtt gtgtggtcca tagtaatcat agaatatagg |
| 5701 | aaaatattaa gacaaagaaa aatagacagg ttaattgata gactaataga aagagcagaa |
| 5761 | gacagtggca atgagagtga aggagaaata tcagcacttg tggagatggg ggtggagatg |
| 5821 | gggcaccatg ctccttggga tgttgatgat ctgtagtgct acagaaaaat tgtgggtcac |
| 5881 | agtctattat ggggtacctg tgtggaagga agcaaccacc actctatttt gtgcatcaga |
| 5941 | tgctaaagca tatgatacag aggtacataa tgtttgggcc acacatgcct gtgtacccac |
| 6001 | agaccccaac ccacaagaag tagtattggt aaatgtgaca gaaaatttta acatgtggaa |
| 6061 | aaatgacatg gtagaacaga tgcatgagga tataatcagt ttatgggatc aaagcctaaa |
| 6121 | gccatgtgta aaattaaccc cactctgtgt tagtttaaag tgcactgatt tgaagaatga |
| 6181 | tactaatacc aatagtagta gcgggagaat gataatggag aaaggagaga taaaaaactg |
| 6241 | ctctttcaat atcagcacaa gcataagagg taaggtgcag aaagaatatg cattttttta |
| 6301 | taaacttgat ataataccaa tagataatga tactaccagc tataagttga caagttgtaa |
| 6361 | cacctcagtc attacacagg cctgtccaaa ggtatccttt gagccaattc ccatacatta |
| 6421 | ttgtgccccg gctggttttg cgattctaaa atgtaataat aagacgttca atggaacagg |
| 6481 | accatgtaca aatgtcagca cagtacaatg tacacatgga attaggccag tagtatcaac |
| 6541 | tcaactgctg ttaaatggca gtctagcaga agaagaggta gtaattagat ctgtcaattt |
| 6601 | cacggacaat gctaaaacca taatagtaca gctgaacaca tctgtagaaa ttaattgtac |
| 6661 | aagacccaac aacaatacaa gaaaaagaat ccgtatccag agaggaccag ggagagcatt |
| 6721 | tgttacaata ggaaaaatag gaaatatgag acaagcacat tgtaacatta gtagagcaaa |
| 6781 | atggaataac actttaaaac agatagctag caaattaaga gaacaatttg gaaataataa |
| 6841 | aacaataatc tttaagcaat cctcaggagg ggacccagaa attgtaacgc acagttttaa |
| 6901 | ttgtggaggg gaatttttct actgtaattc aacacaactg tttaatagta cttggtttaa |
| 6961 | tagtacttgg agtactgaag ggtcaaataa cactgaagga agtgacacaa tcaccctccc |
| 7021 | atgcagaata aaacaaatta taaacatgtg gcagaaagta ggaaaagcaa tgtatgcccc |
| 7081 | tcccatcagt ggacaaatta gatgttcatc aaatattaca gggctgctat taacaagaga |
| 7141 | tggtggtaat agcaacaatg agtccgagat cttcagacct ggaggaggag atatgaggga |
| 7201 | caattggaga agtgaattat ataaatataa agtagtaaaa attgaaccat taggagtagc |
| 7261 | acccaccaag gcaaagagaa gagtggtgca gagagaaaaa agagcagtgg gaataggagc |
| 7321 | tttgttcctt gggttcttgg gagcagcagg aagcactatg ggcgcagcct caatgacgct |
| 7381 | gacggtacag gccagacaat tattgtctgg tatagtgcag cagcagaaca atttgctgag |
| 7441 | ggctattgag gcgcaacagc atctgttgca actcacagtc tggggcatca agcagctcca |
| 7501 | ggcaagaatc ctggctgtgg aaagatacct aaaggatcaa cagctcctgg ggatttgggg |
| 7561 | ttgctctgga aaactcattt gcaccactgc tgtgccttgg aatgctagtt ggagtaataa |
| 7621 | atctctggaa cagatttgga atcacacgac ctggatggag tgggacagag aaattaacaa |
| 7681 | ttacacaagc ttaatacact ccttaattga agaatcgcaa aaccagcaag aaaagaatga |
| 7741 | acaagaatta ttggaattag ataaatgggc aagtttgtgg aattggttta acataacaaa |
| 7801 | ttggctgtgg tatataaaat tattcataat gatagtagga ggcttggtag gtttaagaat |
| 7861 | agtttttgct gtactttcta tagtgaatag agttaggcag ggatattcac cattatcgtt |
| 7921 | tcagacccac ctcccaaccc cgaggggacc cgacaggccc gaaggaatag aagaagaagg |
| 7981 | tggagagaga gacagagaca gatccattcg attagtgaac ggatccttgg cacttatctg |
| 8041 | ggacgatctg cggagcctgt gcctcttcag ctaccaccgc ttgagagact tactcttgat |
| 8101 | tgtaacgagg attgtggaac ttctgggacg cagggggtgg gaagccctca aatattggtg |
| 8161 | gaatctccta cagtattgga gtcaggaact aaagaatagt gctgttagct tgctcaatgc |
| 8221 | cacagccata gcagtagctg aggggacaga tagggttata gaagtagtac aaggagcttg |
| 8281 | tagagctatt cgccacatac ctagaagaat aagacagggc ttggaaagga ttttgctata |
| 8341 | agatgggtgg caagtggtca aaaagtagtg tgattggatg gcctactgta agggaaagaa |
| 8401 | tgagacgagc tgagccagca gcagataggg tgggagcagc atctcgagac ctggaaaaac |
| 8461 | atggagcaat cacaagtagc aatacagcag ctaccaatgc tgcttgtgcc tggctagaag |
| 8521 | cacaagagga ggaggaggtg ggttttccag tcacacctca ggtaccttta agaccaatga |
| 8581 | cttacaaggc agctgtagat cttagccact ttttaaaaga aaagggggga ctggaagggc |
| 8641 | taattcactc ccaaagaaga caagatatcc ttgatctgtg gatctaccac acacaaggct |
| 8701 | acttccctga ttagcagaac tacacaccag ggccaggggt cagatatcca ctgacctttg |
| 8761 | gatggtgcta caagctagta ccagttgagc cagataagat agaagaggcc aataaaggag |
| 8821 | agaacaccag cttgttacac cctgtgagcc tgcatgggat ggatgacccg gagagagaag |
| 8881 | tgttagagtg gaggtttgac agccgcctag catttcatca cgtggcccga gagctgcatc |
| 8941 | cggagtactt caagaactgc tgacatcgag cttgctacaa gggactttcc gctggggact |
| 9001 | ttccagggag gcgtggcctg ggcgggactg gggagtggcg agccctcaga tcctgcatat |
| 9061 | aagcagctgc tttttgcctg tactgggtct ctctggttag accagatctg agcctgggag |
| 9121 | ctctctggct aactagggaa cccactgctt aagcctcaat aaagcttgcc ttgagtgctt |
| 9181 | c |

>Embodiment of stem loop sequence.
(SEQ ID NO: 2; Y = C or T, R = G or A, and W = A)
5′-CUCGGCUUGCUGAAGYGCRCWCRGCAAGAG-3′
>Another embodiment of stem loop sequence
(SEQ ID NO: 3)
5′-CUCGGCUUGCUGAAGCGCGCACGGCAAGAG-3′
>Another embodiment of stem loop sequence as shown in FIG. 4A
(SEQ ID NO: 4)
5′-CUCGGCUUGCUGAAGCGCGCACGGCAAGAGGCGAG-3′
>Another embodiment of stem loop sequence as shown in FIG. 4B
(SEQ ID NO: 5)
5′-CUCGGCUUGCUGAAGCGCGCAGUCGUUCAGGCGAG-3′
>Another embodiment of stem loop sequence as shown in FIG. 4B
(SEQ ID NO: 6)
5′-CUCGGGAACGGCAAGCGCGCACGGCAAGAGGCGAG-3′

SEQ ID NOs: 2-6 above are listed in the sequence listing filed herewith with “T” in place of “U” as shown above as provided by the WIPO ST.26 reference standards (see, for example, Table 1 of WIPO ST.26 Reference Standards, Annex I, Section 1, which provides that “t=thymine in DNA/uracil in RNA (t/u)”).

Although the foregoing has been described in some detail by way of illustration and example, one of skill in the art will appreciate that certain changes and modifications may be practiced within the scope of the appended claims. All publications, patents, and patent applications cited herein are hereby incorporated by reference with respect to the material for which they are cited.

1. A lentiviral vector comprising a first gRNA encoding a first cargo and a second gRNA encoding a second cargo, wherein the first cargo and second cargo are not the same, wherein the first gRNA and second gRNA are not covalently linked, and wherein the first gRNA and second gRNA comprise sequences that reduce or de-stabilize homopairing.

2. The lentiviral vector of claim 1, wherein the first gRNA and second gRNA comprise sequences that increase or promote heteropairing.

3. The lentiviral vector of claim 1, wherein the first gRNA comprises an extended duplex sequence (EDS), wherein the EDS comprises sequences corresponding to the dimer initiation signal (DIS), stem loop packaging sequence, splice donor (“SD”), and the AUG domains of HIV-1.

4. The lentiviral vector of claim 1, wherein the second gRNA comprises an extended duplex sequence (EDS), wherein the EDS comprises sequences corresponding to the DIS, stem loop packaging sequence, splice donor (“SD”), and AUG domains of HIV-1.

5. The lentiviral vector of claim 1 wherein the first gRNA and the second gRNA comprise stem-loop structures, and the stem structure adjacent to a dimer initiation signal (DIS) of the first gRNA and the second gRNA are incompatible.

6-11. (canceled)

12. A lentiviral vector comprising a first gRNA (gRNA^{Long}) encoding a first cargo, wherein the first cargo encoding at least one non-viral polymer is a non-viral polypeptide, a non-viral polynucleotide, a bacterial polypeptide, a bacterial polynucleotide, a eukaryotic polypeptide, a eukaryotic polynucleotide, an animal polypeptide, an animal polynucleotide, a plant polypeptide, a plant polynucleotide, a mammalian polypeptide, a mammalian polynucleotide, a human polypeptide, a human polynucleotide, an antibody, an inhibitory RNA, or a guide RNA, or any combination of any thereof, and a second gRNA (gRNA^{Short}), wherein the first gRNA and second gRNA are not covalently linked, and wherein the second gRNA:
does not encode a polymer selected from a bacterial polypeptide and a bacterial polynucleotide; and/or
does not encode a polymer selected from a eukaryotic polypeptide and a eukaryotic polynucleotide; and/or
does not encode a polymer selected from an animal polypeptide and an animal polynucleotide; and/or
does not encode a polymer selected from a plant polypeptide and a plant polynucleotide; and/or
does not encode a polymer selected from a mammalian polypeptide and a mammalian polynucleotide; and/or
does not encode a polymer selected from a human polypeptide and a human polynucleotide; and/or
does not encode an antibody; and/or
does not encode an inhibitory RNA; and/or
does not encode a guide RNA.

13. The lentiviral vector of claim 12, wherein the second gRNA does not encode a polypeptide that is not a viral polypeptide.

14. The lentivirus vector of claim 13, wherein the second gRNA does not encode a protein other than one or a combination of the following: VSVG, Gag, Rev, Tat, Vpr, Vpx, Vif and Nef.

15. The lentiviral vector of claim 10, wherein the second gRNA does not encode a protein.

16. The lentiviral vector of claim 12, wherein the first gRNA comprises a payload at least 3 times larger, measured in kilobase pairs, than the payload of the second gRNA.

17-21. (canceled)

22. A lentiviral vector comprising a first gRNA (gRNA^{Long}) encoding a first cargo, the first cargo encoding at least one non-lentiviral polymer and is a non-lentiviral polypeptide or a non-lentiviral polynucleotide, and a second gRNA (gRNA^{Short}) encoding a second cargo, wherein the first cargo and second cargo are not the same, wherein the first gRNA and second gRNA are not covalently linked, and wherein the payload flanked by the 5′ and 3′ LTRs of the first gRNA is at least 3 times larger, measured in kilobase pairs, than the payload of the second gRNA.

23-27. (canceled)

28. A lentiviral vector comprising a genome consisting essentially of one single RNA molecule (RNA^{single}).

29. The lentiviral vector of claim 28, wherein the RNA^{single} is greater than about 10 kb in size.

30. The lentiviral vector of claim 29, wherein the RNA^{single} is greater than about 12 kb-18 kb.

31. The lentiviral vector of claim 28, further comprising an upstream packaging signal (PACK 1) and a downstream packaging signal (PACK 2), wherein the PACK 1 and the PACK 2 associate to form a structure recognized by a lentivirus dimerization-dependent packaging mechanism.

32-34. (canceled)

36. A lentivirus particle, comprising a lentiviral vector of claim 1.

37. A kit, comprising:
a lentiviral vector of any one of claim 1, and
instructions for use.

38. A method, comprising:
administering a lentiviral vector according to claim 1 to one or more cells, whereby the administering causes a polynucleotide of the lentiviral vector or lentivirus particle to enter the cell.

39. The method of claim 38, wherein the cell is a mammalian cell.

40. The method of claim 38, wherein the administering is transfection or viral transduction.


SEQUENCE LISTING (6 sequences)

SEQ ID NO: 1 - DNA, 9181 bp, Human immunodeficiency virus 1
  source 1..9181: mol_type = genomic DNA; note = GenBank: AF033819.3
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttc

SEQ ID NO: 2 - RNA, 30 nt, synthetic construct
  source 1..30: mol_type = other RNA; note = Y = C or T; R = G or A; and W = A
ctcggcttgctgaagygcrcwcrgcaagag

SEQ ID NO: 3 - RNA, 30 nt, synthetic construct
  source 1..30: mol_type = other RNA
ctcggcttgctgaagcgcgcacggcaagag

SEQ ID NO: 4 - RNA, 35 nt, synthetic construct
  source 1..35: mol_type = other RNA
ctcggcttgctgaagcgcgcacggcaagaggcgag

SEQ ID NO: 5 - RNA, 35 nt, synthetic construct
  source 1..35: mol_type = other RNA
ctcggcttgctgaagcgcgcagtcgttcaggcgag

SEQ ID NO: 6 - RNA, 35 nt, synthetic construct
  source 1..35: mol_type = other RNA
ctcgggaacggcaagcgcgcacggcaagaggcgag